SEQUENCE LISTING (52 sequences)

SEQ ID NO: 1 - DNA, 375 bp, Artificial Sequence
  Synthetic death marker binding domain
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcgcgattagcggcagcggcggcagcacctattatgcggatagcgtgaaaggccgctttaccattagccgcgataacagcaaaaacaccctgtatctgcagatgaacagcctgcgcgcggaagataccgcggtgtattattgcgcgaaaggccaggtgctgtattatggcagcggcagctatcattggtttgatccgtggggccagggcaccctggtgaccgtgagcagc

SEQ ID NO: 2 - DNA, 375 bp, Artificial Sequence
  Synthetic death marker binding domain
gargtncarytnytngarwsnggnggnggnytngtncarccnggnggnwsnytnmgnytnwsntgygcngcnwsnggnttyacnttywsnwsntaygcnatgwsntgggtnmgncargcnccnggnaarggnytngartgggtnwsngcnathwsnggnwsnggnggnwsnacntaytaygcngaywsngtnaarggnmgnttyacnathwsnmgngayaaywsnaaraayacnytntayytncaratgaaywsnytnmgngcngargayacngcngtntaytaytgygcnaarggncargtnytntaytayggnwsnggnwsntaycaytggttygayccntggggncarggnacnytngtnacngtnwsnwsn

SEQ ID NO: 3 - RNA, 375 nt, Artificial Sequence
  Synthetic death marker binding domain
gaagugcagcugcuggaaagcggcggcggccuggugcagccgggcggcagccugcgccugagcugcgcggcgagcggcuuuaccuuuagcagcuaugcgaugagcugggugcgccaggcgccgggcaaaggccuggaaugggugagcgcgauuagcggcagcggcggcagcaccuauuaugcggauagcgugaaaggccgcuuuaccauuagccgcgauaacagcaaaaacacccuguaucugcagaugaacagccugcgcgcggaagauaccgcgguguauuauugcgcgaaaggccaggugcuguauuauggcagcggcagcuaucauugguuugauccguggggccagggcacccuggugaccgugagcagc

SEQ ID NO: 4 - protein, 125 aa, Artificial Sequence
  Synthetic death marker binding domain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGQVLYYGSGSYHWFDPWGQGTLVTVSS

SEQ ID NO: 5 - DNA, 327 bp, Artificial Sequence
  Synthetic death marker binding domain
gatattcagatgacccagagcccgagcagcctgagcgcgagcgtgggcgatcgcgtgaccattacctgccgcgcgagccagggcattcgcaacgatctgggctggtatcagcagaaaccgggcaaagcgccgaaacgcctgatttatgcggcgagcagcctggaaagcggcgtgccgagccgctttagcggcagcggcagcggcaccgaatttaccctgaccattagcagcctgcagccggaagattttgcgacctattattgcctgcagcataacagctatccgctgacctttggcggcggcaccaaagtggaaattaaacgcacc

SEQ ID NO: 6 - DNA, 327 bp, Artificial Sequence
  Synthetic death marker binding domain
gayathcaratgacncarwsnccnwsnwsnytnwsngcnwsngtnggngaymgngtnacnathacntgymgngcnwsncarggnathmgnaaygayytnggntggtaycarcaraarccnggnaargcnccnaarmgnytnathtaygcngcnwsnwsnytngarwsnggngtnccnwsnmgnttywsnggnwsnggnwsnggnacngarttyacnytnacnathwsnwsnytncarccngargayttygcnacntaytaytgyytncarcayaaywsntayccnytnacnttyggnggnggnacnaargtngarathaarmgnacn

SEQ ID NO: 7 - RNA, 326 nt, Artificial Sequence
  Synthetic death marker binding domain
auauucagaugacccagagcccgagcagccugagcgcgagcgugggcgaucgcgugaccauuaccugccgcgcgagccagggcauucgcaacgaucugggcugguaucagcagaaaccgggcaaagcgccgaaacgccugauuuaugcggcgagcagccuggaaagcggcgugccgagccgcuuuagcggcagcggcagcggcaccgaauuuacccugaccauuagcagccugcagccggaagauuuugcgaccuauuauugccugcagcauaacagcuauccgcugaccuuuggcggcggcaccaaaguggaaauuaaacgcacc

SEQ ID NO: 8 - protein, 109 aa, Artificial Sequence
  Synthetic death marker binding domain
DIQMTQSPSSLSASVGDRVTITCRASQGIRNDLGWYQQKPGKAPKRLIYAASSLESGVPSRFSGSGSGTEFTLTISSLQPEDFATYYCLQHNSYPLTFGGGTKVEIKRT

SEQ ID NO: 9 - DNA, 333 bp, Artificial Sequence
  Synthetic death marker binding domain
gatattcagatgacccagagcccgagcagcctgagcgcgagcgtgggcgatcgcgtgaccattagctgcaccggcccgagcagcccggtgggcggctataaaccgattagctggtatcatcagcatccgggcaccgcgccgaaactgatgatttatattcgcggcatgcagcgcagcggcgtgccggatcgctttagcggcagcaaaagcggcaacaccgcgagcctgaccattagcggcctgcgcgcgatggatgaagcggattattattgcgcgcagtatgatgaactgccgtatacctttggccagggcaccaaactggaagtgaaacgc

SEQ ID NO: 10 - DNA, 333 bp, Artificial Sequence
  Synthetic death marker binding domain
gayathcaratgacncarwsnccnwsnwsnytnwsngcnwsngtnggngaymgngtnacnathwsntgyacnggnccnwsnwsnccngtnggnggntayaarccnathwsntggtaycaycarcayccnggnacngcnccnaarytnatgathtayathmgnggnatgcarmgnwsnggngtnccngaymgnttywsnggnwsnaarwsnggnaayacngcnwsnytnacnathwsnggnytnmgngcnatggaygargcngaytaytaytgygcncartaygaygarytnccntayacnttyggncarggnacnaarytngargtnaarmgn

SEQ ID NO: 11 - RNA, 333 nt, Artificial Sequence
  Synthetic death marker binding domain
gauauucagaugacccagagcccgagcagccugagcgcgagcgugggcgaucgcgugaccauuagcugcaccggcccgagcagcccggugggcggcuauaaaccgauuagcugguaucaucagcauccgggcaccgcgccgaaacugaugauuuauauucgcggcaugcagcgcagcggcgugccggaucgcuuuagcggcagcaaaagcggcaacaccgcgagccugaccauuagcggccugcgcgcgauggaugaagcggauuauuauugcgcgcaguaugaugaacugccguauaccuuuggccagggcaccaaacuggaagugaaacgc

SEQ ID NO: 12 - protein, 111 aa, Artificial Sequence
  Synthetic death marker binding domain
DIQMTQSPSSLSASVGDRVTISCTGPSSPVGGYKPISWYHQHPGTAPKLMIYIRGMQRSGVPDRFSGSKSGNTASLTISGLRAMDEADYYCAQYDELPYTFGQGTKLEVKR

SEQ ID NO: 13 - DNA, 375 bp, Artificial Sequence
  Synthetic death marker binding domain
caggtgatgcagctggtggaaagcggcggcggcctggtgcagccgggccgcagcctgcgcctgagctgcgcggcgagcggctttacctttaacgattatccgctgcattgggtgcgccagccgccgggcaaaggcctggaatggagcagcggcattagctggaacagcggcagcattggctatgcggatagcgtgaaaggccgctttaccattagccgcgataacgcgaaaaacagcctgtatctgcagatgaacagcctgcgcgcggaagataccgcgctgtattattgcgcgaaaggcccgccgggctattatgatagcagcgaaccgagcgattggggccagggccatggccatctggtgaccgtgagcagc

SEQ ID NO: 14 - DNA, 375 bp, Artificial Sequence
  Synthetic death marker binding domain
cargtnatgcarytngtngarwsnggnggnggnytngtncarccnggnmgnwsnytnmgnytnwsntgygcngcnwsnggnttyacnttyaaygaytayccnytncaytgggtnmgncarccnccnggnaarggnytngartggwsnwsnggnathwsntggaaywsnggnwsnathggntaygcngaywsngtnaarggnmgnttyacnathwsnmgngayaaygcnaaraaywsnytntayytncaratgaaywsnytnmgngcngargayacngcnytntaytaytgygcnaarggnccnccnggntaytaygaywsnwsngarccnwsngaytggggncarggncayggncayytngtnacngtnwsnwsn

SEQ ID NO: 15 - RNA, 375 nt, Artificial Sequence
  Synthetic death marker binding domain
caggugaugcagcugguggaaagcggcggcggccuggugcagccgggccgcagccugcgccugagcugcgcggcgagcggcuuuaccuuuaacgauuauccgcugcauugggugcgccagccgccgggcaaaggccuggaauggagcagcggcauuagcuggaacagcggcagcauuggcuaugcggauagcgugaaaggccgcuuuaccauuagccgcgauaacgcgaaaaacagccuguaucugcagaugaacagccugcgcgcggaagauaccgcgcuguauuauugcgcgaaaggcccgccgggcuauuaugauagcagcgaaccgagcgauuggggccagggccauggccaucuggugaccgugagcagc

SEQ ID NO: 16 - protein, 125 aa, Artificial Sequence
  Synthetic death marker binding domain
QVMQLVESGGGLVQPGRSLRLSCAASGFTFNDYPLHWVRQPPGKGLEWSSGISWNSGSIGYADSVKGRFTISRDNAKNSLYLQMNSLRAEDTALYYCAKGPPGYYDSSEPSDWGQGHGHLVTVSS

SEQ ID NO: 17 - DNA, 333 bp, Artificial Sequence
  Synthetic death marker binding domain
gatattcagatgacccagagcccgagcagcctgagcgcgagcgtgggcgatcgcgtgaccattacctgcagcggcgataaactgggcgataaatatgcgtgctggtatcagcagaaaccgggccagagcccggtgctggtgatttatcaggatagcaaacgcccgagcggcattccggaacgctttagcggcagcaacagcggcaacaccgcgaccctgaccattagcggcacccaggcgatggatgaagcggattattattgcaacgcgtgggatagcagcacctatgtggtgtttggcggcggcaccaaactgaccgtgctgggccagccg

SEQ ID NO: 18 - DNA, 333 bp, Artificial Sequence
  Synthetic death marker binding domain
gayathcaratgacncarwsnccnwsnwsnytnwsngcnwsngtnggngaymgngtnacnathacntgywsnggngayaarytnggngayaartaygcntgytggtaycarcaraarccnggncarwsnccngtnytngtnathtaycargaywsnaarmgnccnwsnggnathccngarmgnttywsnggnwsnaaywsnggnaayacngcnacnytnacnathwsnggnacncargcnatggaygargcngaytaytaytgyaaygcntgggaywsnwsnacntaygtngtnttyggnggnggnacnaarytnacngtnytnggncarccn

SEQ ID NO: 19 - RNA, 333 nt, Artificial Sequence
  Synthetic death marker binding domain
gauauucagaugacccagagcccgagcagccugagcgcgagcgugggcgaucgcgugaccauuaccugcagcggcgauaaacugggcgauaaauaugcgugcugguaucagcagaaaccgggccagagcccggugcuggugauuuaucaggauagcaaacgcccgagcggcauuccggaacgcuuuagcggcagcaacagcggcaacaccgcgacccugaccauuagcggcacccaggcgauggaugaagcggauuauuauugcaacgcgugggauagcagcaccuaugugguguuuggcggcggcaccaaacugaccgugcugggccagccg

SEQ ID NO: 20 - protein, 111 aa, Artificial Sequence
  Synthetic death marker binding domain
DIQMTQSPSSLSASVGDRVTITCSGDKLGDKYACWYQQKPGQSPVLVIYQDSKRPSGIPERFSGSNSGNTATLTISGTQAMDEADYYCNAWDSSTYVVFGGGTKLTVLGQP

SEQ ID NO: 21 - DNA, 375 bp, Artificial Sequence
  Synthetic death marker binding domain
gaagtgcagctggtggaaagcggcggcggcctggtgcagccgggcggcagcctgcgcctgagctgcgcggcgagcggctttacctttagcagctatagcatgagctgggtgcgccaggcgccgggcaaaggcctggaatggattagctatattagcagcagcagcagcaccatttattatgcggatagcgtgaaaggccgctttaccattagccgcgataacgcgaaaaacagcctgtatctgcagatgaacagcctgcgcgcggaagataccgcggtgtattattgcgcgcgcagccgcaactatgatagcagcggctattatagccattattttgatttttggggccagggcaccatggtgaccgtgagcagc

SEQ ID NO: 22 - DNA, 375 bp, Artificial Sequence
  Synthetic death marker binding domain
gargtncarytngtngarwsnggnggnggnytngtncarccnggnggnwsnytnmgnytnwsntgygcngcnwsnggnttyacnttywsnwsntaywsnatgwsntgggtnmgncargcnccnggnaarggnytngartggathwsntayathwsnwsnwsnwsnwsnacnathtaytaygcngaywsngtnaarggnmgnttyacnathwsnmgngayaaygcnaaraaywsnytntayytncaratgaaywsnytnmgngcngargayacngcngtntaytaytgygcnmgnwsnmgnaaytaygaywsnwsnggntaytaywsncaytayttygayttytggggncarggnacnatggtnacngtnwsnwsn

SEQ ID NO: 23 - RNA, 375 nt, Artificial Sequence
  Synthetic death marker binding domain
gaagugcagcugguggaaagcggcggcggccuggugcagccgggcggcagccugcgccugagcugcgcggcgagcggcuuuaccuuuagcagcuauagcaugagcugggugcgccaggcgccgggcaaaggccuggaauggauuagcuauauuagcagcagcagcagcaccauuuauuaugcggauagcgugaaaggccgcuuuaccauuagccgcgauaacgcgaaaaacagccuguaucugcagaugaacagccugcgcgcggaagauaccgcgguguauuauugcgcgcgcagccgcaacuaugauagcagcggcuauuauagccauuauuuugauuuuuggggccagggcaccauggugaccgugagcagc

SEQ ID NO: 24 - protein, 125 aa, Artificial Sequence
  Synthetic death marker binding domain
EVQLVESGGGLVQPGGSLRLSCAASGFTFSSYSMSWVRQAPGKGLEWISYISSSSSTIYYADSVKGRFTISRDNAKNSLYLQMNSLRAEDTAVYYCARSRNYDSSGYYSHYFDFWGQGTMVTVSS

SEQ ID NO: 25 - DNA, 354 bp, Artificial Sequence
  Synthetic death marker binding domain
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcgcgattagcggcagcggcggcagcacctattatgcggatagcgtgaaaggccgctttaccattagccgcgataacagcaaaaacaccctgtatctgcagatgaacagcctgcgcgcggaagataccgcggtgtattattgcgcgaaagatggcgaatatgaagcgggcattgattattggtttgatccgtggggccagggcaccctg

SEQ ID NO: 26 - DNA, 354 bp, Artificial Sequence
  Synthetic death marker binding domain
gargtncarytnytngarwsnggnggnggnytngtncarccnggnggnwsnytnmgnytnwsntgygcngcnwsnggnttyacnttywsnwsntaygcnatgwsntgggtnmgncargcnccnggnaarggnytngartgggtnwsngcnathwsnggnwsnggnggnwsnacntaytaygcngaywsngtnaarggnmgnttyacnathwsnmgngayaaywsnaaraayacnytntayytncaratgaaywsnytnmgngcngargayacngcngtntaytaytgygcnaargayggngartaygargcnggnathgaytaytggttygayccntggggncarggnacnytn

SEQ ID NO: 27 - RNA, 354 nt, Artificial Sequence
  Synthetic death marker binding domain
gaagugcagcugcuggaaagcggcggcggccuggugcagccgggcggcagccugcgccugagcugcgcggcgagcggcuuuaccuuuagcagcuaugcgaugagcugggugcgccaggcgccgggcaaaggccuggaaugggugagcgcgauuagcggcagcggcggcagcaccuauuaugcggauagcgugaaaggccgcuuuaccauuagccgcgauaacagcaaaaacacccuguaucugcagaugaacagccugcgcgcggaagauaccgcgguguauuauugcgcgaaagauggcgaauaugaagcgggcauugauuauugguuugauccguggggccagggcacccug

SEQ ID NO: 28 - protein, 118 aa, Artificial Sequence
  Synthetic death marker binding domain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGEYEAGIDYWFDPWGQGTL

SEQ ID NO: 29 - DNA, 321 bp, Artificial Sequence
  Synthetic death marker binding domain
gatattcagatgacccagagcccgagcagcctgagcgcgagcgtgggcgatcgcgtgaccattacctgccgcagcagccaggatattaacaaatatattggctggtatcagcagaaaccgggcaaagcgccgaaactgctgattcattataccagcaccctgcagccgggcgtgccgagccgctttagcggcagcggcagcggcaccgattttaccctgaccattagcagcctgcagccggaagattttgcgacctatttttgcctgaactatgataacctgtatagctttggcggcggcaccaaagtggaaattaaacgc

SEQ ID NO: 30 - DNA, 321 bp, Artificial Sequence
  Synthetic death marker binding domain
gayathcaratgacncarwsnccnwsnwsnytnwsngcnwsngtnggngaymgngtnacnathacntgymgnwsnwsncargayathaayaartayathggntggtaycarcaraarccnggnaargcnccnaarytnytnathcaytayacnwsnacnytncarccnggngtnccnwsnmgnttywsnggnwsnggnwsnggnacngayttyacnytnacnathwsnwsnytncarccngargayttygcnacntayttytgyytnaaytaygayaayytntaywsnttyggnggnggnacnaargtngarathaarmgn

SEQ ID NO: 31 - RNA, 321 nt, Artificial Sequence
  Synthetic death marker binding domain
gauauucagaugacccagagcccgagcagccugagcgcgagcgugggcgaucgcgugaccauuaccugccgcagcagccaggauauuaacaaauauauuggcugguaucagcagaaaccgggcaaagcgccgaaacugcugauucauuauaccagcacccugcagccgggcgugccgagccgcuuuagcggcagcggcagcggcaccgauuuuacccugaccauuagcagccugcagccggaagauuuugcgaccuauuuuugccugaacuaugauaaccuguauagcuuuggcggcggcaccaaaguggaaauuaaacgc

SEQ ID NO: 32 - protein, 107 aa, Artificial Sequence
  Synthetic death marker binding domain
DIQMTQSPSSLSASVGDRVTITCRSSQDINKYIGWYQQKPGKAPKLLIHYTSTLQPGVPSRFSGSGSGTDFTLTISSLQPEDFATYFCLNYDNLYSFGGGTKVEIKR

SEQ ID NO: 33 - protein, 2 aa, Artificial Sequence
  Synthetic peptide metal binding domain
GC

SEQ ID NO: 34 - protein, 3 aa, Artificial Sequence
  Synthetic peptide metal binding domain
GRC

SEQ ID NO: 35 - protein, 3 aa, Artificial Sequence
  Synthetic peptide metal binding domain
GKC

SEQ ID NO: 36 - protein, 5 aa, Artificial Sequence
  Synthetic peptide metal binding domain
GDGRC

SEQ ID NO: 37 - protein, 5 aa, Artificial Sequence
  Synthetic peptide metal binding domain
GEGRC

SEQ ID NO: 38 - protein, 5 aa, Artificial Sequence
  Synthetic peptide metal binding domain
GDGKC

SEQ ID NO: 39 - protein, 5 aa, Artificial Sequence
  Synthetic peptide metal binding domain
GEGKC

SEQ ID NO: 40 - protein, 5 aa, Artificial Sequence
  Synthetic peptide metal binding domain
EEEEE

SEQ ID NO: 41 - protein, 6 aa, Artificial Sequence
  Synthetic peptide metal binding domain
EEEEEE

SEQ ID NO: 42 - protein, 5 aa, Artificial Sequence
  Synthetic peptide metal binding domain
DDDDD

SEQ ID NO: 43 - protein, 6 aa, Artificial Sequence
  Synthetic peptide metal binding domain
DDDDDD

SEQ ID NO: 44 - protein, 11 aa, Artificial Sequence
  Synthetic peptide metal binding domain
FHCPYDLCHIL

SEQ ID NO: 45 - protein, 5 aa, Artificial Sequence
  Synthetic peptide metal binding domain
GDGRH

SEQ ID NO: 46 - protein, 5 aa, Artificial Sequence
  Synthetic peptide metal binding domain
GEGRH

SEQ ID NO: 47 - protein, 5 aa, Artificial Sequence
  Synthetic peptide metal binding domain
GDGKH

SEQ ID NO: 48 - protein, 5 aa, Artificial Sequence
  Synthetic peptide metal binding domain
GEGKH

SEQ ID NO: 49 - protein, 3 aa, Artificial Sequence
  Synthetic peptide metal binding domain
GRH

SEQ ID NO: 50 - protein, 3 aa, Artificial Sequence
  Synthetic peptide metal binding domain
GKH

SEQ ID NO: 51 - DNA, 13 bp, Artificial Sequence
  Synthetic primer
ggccnnnnnggcc

SEQ ID NO: 52 - DNA, 6 bp, Artificial Sequence
  Synthetic primer
ccgcgg

(19) United States
(12) Patent Application Publication
(10) Pub. No.: US 2012/0093730 A1
(43) Pub. Date: Apr. 19, 2012

(54) MOLECULAR DEATH TAGS AND METHODS OF THEIR USE

(76) Inventors: Marek Malecki, Pomona, CA (US); Raf Malecki, San Francisco, CA (US); Bianca Malecki, Madison, WI (US)

(21) Appl. No.: 13/267,823

(22) Filed: Oct. 6, 2011

Related U.S. Application Data
(60) Provisional application No. 61/390,292, filed on Oct. 6, 2010.

(51) Int. Cl. A61K 49/00; C07K 16/18; B82Y 15/00

(52) U.S. Cl. 424/9.2; 530/391.3; 977/810; 977/838; 977/902

(57) ABSTRACT

In one embodiment, a death tag for targeting a cell death marker is provided, the death tag comprising a death marker binding domain; a reporter binding domain (RBD); and a reporter component that is associated with the reporter binding domain. In another embodiment, a method of determining the efficacy of a cancer treatment is provided. The method may comprise administering to a subject an effective dose of a death tag that targets apoptotic, necrotic or dead cells; exposing the subject to an imaging technique; determining that the cancer treatment is effective when the imaging technique detects the presence of the death tag. In another embodiment, an in vivo, ex vivo, or in vitro method of determining the need for a treatment or determining the efficacy of a treatment in cell, tissue, and organ injuries.

### CROSS-REFERENCE TO RELATED APPLICATIONS

This application claims the benefit of U.S. Provisional Patent Application No. 61/390,292, filed Oct. 6, 2010, which is hereby incorporated by reference in its entirety, as if fully set forth herein.

### BACKGROUND

Many cancers are diagnosed in later stages of the disease because of low sensitivity of existing diagnostic procedures and processes. More than 1.5 million people will be newly diagnosed with cancer this year (Jemal et al. 2010), almost 600,000 people will die of cancer in the USA in 2010. Millions harbor early-stage cancer without knowing it. Cancer is the number one killer of people under 85.

In 2010, the National Cancer Institute estimates that in the United States nearly 200,000 women will be diagnosed with breast cancer and over 40,000 women will die of breast cancer. These tragic statistics are largely a result of late diagnoses and inefficient treatments having deleterious side effects.

Survival statistics that exist for many types of cancer are bleak. The 5 year survival for women diagnosed with stage I ovarian cancer (limited to ovaries) reaches 90%, but for women diagnosed with stage IV ovarian cancer (metastasized to distant organs) 5 year survival falls below 5% (Jemal et al. 2010). More than 80% of women diagnosed with ovarian cancer are diagnosed with malignant ovarian cancer. Presently, there is no screening program for women highly susceptible to acquire ovarian cancer, nor is there a method to detect metastasizing cancer cells in their blood or lymph. While many of the metastasizing cancer cells are eliminated by the immune system's natural killer cells (NKC), it only takes one metastatic cell that is not eliminated to give a rise to a malignant, metastatic tumor remaining undetected until it is too late.

Prostate and lung cancer also have bleak survival statistics for patients with metastatic disease. Nearly 100% of patients diagnosed with stage 1 prostate cancer survive 5 years. However, as soon as the prostate cancer reaches stage III, the 5 year survival drops to 50%. The 5 year survival rate for stage 1 lung cancer patients is 50%, but stage IV patients have a 95% mortality rate over 5 years. Therefore, monitoring the progress of metastatic cancer is an important element of the oncological care.

Successful diagnosis and treatment of neoplasms are contingent upon detecting onset of a neoplasm at its earliest stage, so that therapies can be immediately implemented. The earliest discovery of metastases is also important. Once a neoplasm has been detected, a course of therapy or treatment is selected. Upon early detection of metastasis, physicians may be able to provide better more effective treatments before cancers become too advanced for effective treatment.

Common courses of cancer therapy or treatment include surgery, radiation therapy and chemotherapy. Targeted therapies such as immunotherapy, radioimmunotherapy and gene therapy are also increasingly common. Success of these targeted therapies is dependent on early detection and efficient targeting of antibodies to cancer biomarkers that are constantly changing. Once a primary cancer becomes invasive and metastatic, the invading cells or small population of metastasizing cells may escape detection and may become a source of relapse. These small populations of metastasizing cells require the use of toxic, systemic therapy such as chemotherapy and radiation therapy.

Traditional cancer treatments and systemic therapies carry serious side effects. Such therapies are aimed at inducing death of cancer cells, but they also may injure or damage healthy cells. Hence, a delicate balance between killing cancer cells and injuring, damaging or killing healthy cells must be monitored by the ratio between beneficial and non-beneficial effects of a therapy and by adjusting the therapeutic regimes accordingly. Therefore, determining the efficacy as early as possible is important to spare a patient from unnecessary suffering from an ineffective therapy or treatment.

Determining a therapy or treatment's efficacy can be difficult because not all tumors respond to a particular therapy in the same manner. For example, some tumors are very sensitive to radiation, requiring smaller doses of radiation. Other tumors, however, are resistant to radiation. In such cases, one or more alternative therapies should be pursued, rather than continuing an ineffective, harmful course of treatment. Similarly, immunotherapy can be very effective in some well targeted approaches. However, if the tumor does not express the antibody's target receptor or the antibodies are not exclusively specific to cancer cells (e.g., Avastin®), treatment with the immunotherapy may not be beneficial because the harmful side effects could outweigh the negligible beneficial effects of the treatment.

Typically, the efficacy of a chosen therapy is measured by determining a change of tumor size, or lack thereof, via imaging methods such as magnetic resonance imaging (MRI), ultrasonography (USG) and computed tomography (CT). A decrease in tumor size indicates tumor regression and success of a treatment. On the other hand, no change or an increase in tumor size is indicative of therapeutic failure.

Detectable changes in tumor size as a result of a particular therapy usually do not show up in imaging methods for weeks. Continuing a therapy for such an extended time without an indication of whether the therapy is working not only exposes a patient to the harmful effect of a treatment without knowing whether it is also killing cancer cells, but if the therapy is ineffective, it allows the cancer to progress further, putting the patient at risk for further invasion and metastasis.

Therefore, it would be advantageous to develop processes or procedures that can (i) determine whether treated cells are dead or in the process of dying in order to validate progression or regression of the disease and to (ii) determine the effectiveness of a selected therapy or treatment regimen or its side effects in a timely fashion to reduce or prevent unnecessary damage to healthy cells from ineffective therapy or treatment regimens.

Such processes or procedures would also be advantageous for assessing or determining the extent of injury or cell death, assessing the need for immediate therapeutic intervention and evaluating the effectiveness of a particular therapeutic intervention in injuries, diseases or conditions other than cancer, such as those resulting from crushed or damaged organs, tissues, and cells in accidents, explosions (including IEDs), sport injuries.

### SUMMARY

In one embodiment, a death tag for targeting a molecule or process that is accessible, developing, or present, and which is or becomes a manifestation of cell death is provided, the death tag comprising a death marker binding domain; a reporter binding domain (RBD); and a reporter component that is associated with the reporter binding domain. The death tag may be indicative of all types and stages of early apoptosis or necrosis, cell damage, cell disruption, or ultimate cell death. In some embodiments, the death marker may be genomic DNA, single-stranded DNA, double stranded DNA, lamins, histones, nuclear matrix molecules, cell cytoskeleton molecules, contractile molecules, microsomes, or fragments thereof. In some embodiments, the death marker binding domain is a single chain variable fragment (scFv), single domain variable fragment (sdFv), CDR fragment, SDR fragment, CD fragment, Fab fragment, IgG fragment, Fab2 fragment, or IgM fragment.

In some embodiments, the reporter component is a noble metal nanoparticle, which may be selected from the group of Au, Pt, Pd and Ag. In other embodiments, the metal nanoparticle tag is a superparamagnetic metal nanoparticle, which may be selected from the group of Gd, Eu, Fe, Ni, or Co. In other embodiments, the metal nanoparticle tag is a core-shell nanoparticle, the core shell nanoparticle comprising an inner superparamagnetic metal core and an outer noble metal shell.

In some embodiments, the death tag can be used to detect the extent of cell death resulting from toxic cancer treatment, pathological conditions or diseases, or trauma related cell death resulting from myocardial infarction, stroke, frost, heat, ischemia, traffic accidents, battle field injuries, or other causes of cell death. In other embodiments, the death tag can be used to target and remove circulating free DNA from a physiological fluid in the subject (e.g., in cancer, traumatic tissue damage, or Lupus Erythomatosus).

In another embodiment, a method of determining the efficacy of a cancer treatment is provided. The method may comprise administering to a subject an effective dose of a death tag that targets apoptotic, necrotic or dead cells; exposing the subject to an imaging technique; determining that the cancer treatment is effective when the imaging technique detects the presence of the death tag the targeted cells. In some embodiments, the death tag used in the method may be a death tag as described above. In other embodiments, the diagnostic imaging technique is x-ray radiography, computed tomography (CT), magnetic resonance imaging (MRI), Raman, gamma scintigraphy, Raman spectral imaging (RSI), positron emission tomography (PET), single photon emission computed tomography (SPECT), ultrasonography (USG) and fluorescence imaging (e.g., fluorescein (FL) or FL-derivative imaging).

### BRIEF DESCRIPTION OF THE DRAWINGS

FIG. 1 is a representative image of a cancer phantom of several cancer cell lines provided by ATCC that were radiated with 1 Gy. The cells were dispensed into multi-well plates, therapeutically treated, then labeled with the death-tags manufactured for CT according to the protocols described herein, thoroughly rinsed, and imaged with computed tomography (CT). The cells were grown in the wells of multiwell plates within the extracellular matrix (ECM) and immersed within the ECM. The upper row from left to right were: U87 cells (well 1), Brain Cancer DBTRG-05MG cells (well 2), MCF7 cells (well 3), Ovarian cancer OVCAR3 cells (well 4), matrix with serum (control, well 5), matrix with medium (control, well 6) and matrix with PBS (control, well 7). The lower row from left to right were: CRL-1469 cells (well 1), Carcinoma of pancreas CRL-1420 (well 2), HTB-180 cells (well 3), Carcinoma of lungs HTB-173 (well 4), Carcinoma of Testis Tera-1 (well 5), 2 pellets of SKBR cells loaded into the well just prior to labeling with no radiation, chemotherapy, or permeabilization (healthy cell control, well 6) and 1 pellet of SKBR cells permeabilized with Tritonx100 and mixed with matrix (well 7). Intensity of the wells' brightness in the spots reflects intensity of death.

FIG. 2 is a representative image of a cancer phantom of several cancer cell lines provided by ATCC that were treated with chemotherapy. The cells were dispensed into multi-well plates, therapeutically treated, then labeled with the death-tags manufactured for MRI according to the protocols described herein, thoroughly rinsed, and imaged with Magnetic Resonance Imaging (MRI). The cells were grown in the wells of multiwell plates within extracellular matrix (ECM) and immersed within the ECM. The wells were: from right to left: carcinoma of pancreas cells (CRL-1429) (1), carcinoma of testis cells (Tera-1) (2), brain cancer cells (DBTRG-05MG) (3), ovarian cancer cells (OVCAR3) (4) and carcinoma of lung cells (HTB-173) (5). Intensity of the wells' brightness in the spots reflects intensity of death.

FIG. 3 shows a nude mouse suffering from cancer and treated with an antioxidant enzyme blocker that induces cancer cell suicide (e.g., antiSOD, anti-Gpx, anti-caspase or a combination thereof). Effectiveness of the therapy was evaluated by intra-tail-vein injection of death-tags and imaged with fluorescence. Body heat is captured at the red channel revealing the whole body anatomy. Death tags labeling the dead cells are shown in green channel (arrow) highlighting the dead cancer cells due to the cancer suicide therapy.

FIG. 4 shows the workflow for separating and isolating DNA from cancer cells. The mixture of normal (smaller yellow) and ovarian cancer (bigger yellow) cells are collected into the spin tubes (a). The cells are labeled with the fluorescent or superparamagnetic or fluorescent scFv targeting the epidermal growth factor receptor variant III mutation (EGFRvIII) (bigger cells painted red cells), which are present only on the cancer cells (b). The EGFRvIII positive cells are sorted one cell per well either by magnetically activated sorting (MAS) or fluorescently activated sorting (FAS) (d). This is followed by adding hypotonic solutions to swell the cells (d). The membranes and skeletons in swollen cells are broken with the ultrasonic waves. The chromosomes disperse from each cancer cell (e). The chromosomes labeled with the scFv targeting dsDNA are sorted one chromosome per well, picked onto the filmed grids for imaging or unraveled for sequencing (f).

FIG. 5 shows an isolated ovarian cancer cell labeled with fluorescent anti-EGFRvIII scFv and imaged upon excitation with the tunable, pulsed-laser within multi-photon microscopy. Cells from peritoneal washing were labeled with fluorescent single chain variable fragment antibodies targeting epidermal growth factor receptor variant III mutation_{I} (f*scFv_{EGFRvIII}) and sorted using fluorescent activated cell sorter (FACS). Chelating metal binding domains with europium rendered scFv fluorescent. Fluorescence was emitted at the far red peak of 619 nm was very stable upon excitation with the pulsed laser within the multi-photon, fluorescence microscope. Horizontal field width (HFW): 45 μm.

FIG. 6 shows an EGFRvIII positive cell lysate labeled with scFv_{EGFRvIII}. Validation of fluorescent scFv targeting with high specificity EGFRvIII. The blot illustrates: OVCAR 3 cells from ATCC serving as controls (a,b) and ovarian cancer cells (c,d). The OVCAR3 cells expressed EGFR wild type (EGFRwt), but not EGFRvIII (a). The OVCAR3, which was transfected with the EGFRvIII coding sequence from the plasmid obtained from ATCCC, was expressing the mutation EGFR variant III (EGFRvIII) (b). The ovarian cancer cell from the patient diagnosed with the EGFRwt, but not EGFRvIII (c). The cells from the cells of the patient diagnosed with EGFRvIII mutation were strongly expressing EGFRvIII (d). The isolated cell lysates were electrophoresed and electrotransferred. They were amplified and labeled with the EGFRvIII biotags-fluorescent scFvs targeting EGFRvIII (f*scFv_{EGFRvIII}). The gene expression products from the DNA plasmids constructs, received from ATCC, coding wild type and truncated gene expression products served as the type III mutant negative—no band after labeling with F*scFv_{EGFRvIII}(a) and positive controls—strong, single band after labeling with F*scFv_{EGFRvIII} (b). The cell lysates allowed us to identify the EGFRvIII negative (FIG. 3c) and positive (d) ovarian cancer cells on the immunoblots. Please notice very specific labeling attained with out f*scFvEGFRvIII, as demonstrated by the very clean background with no other molecules labeled.

FIG. 7 shows RT PCR of the EGFRvIII positive cell. The additional validation of the correct targeting of the EGFRvIII positive cells with the F*scFv_{EGFRvIII}. Total RNA was isolated, reverse transcribed and amplified by polymerase chain reaction. The coding sequences for EGFR (a) and EGFRvIII (b) from the DNA plasmid constructs received from ATCC were amplified by polymerase chain reaction to serve as the controls. The single ovarian cancer cell was sorted on FACS. Thereafter, mRNA was reverse transcribed into cDNA and amplified by PCR (c). The amplicons allowed us to identify the transcripts of EGFRwt (c) and EGFRvIII (d) in the ovarian cancer cells on the gels. Noted are the specific amplifications demonstrated by the clean background.

FIG. 8 shows FISH of the centromeric and EGFR probes on the isolated chromosome 7. The selected chromosome was hybridized with the probes annealing with the centromeric DNA (green) and EGFR (red) and labeled with the scFv targeting dsDNA (blue). The centromere was labeled by the fluorescent in situ hybridization (FISH) with the probe modified with the biotin, which was then labeled with the FISH with the probe modified with the scFv targeting biotin and chelating Tb (green marker). The locus for the epidermal growth factor receptor coding sequence was labeled with the probe modified with digoxigenin, which was then labeled with the scFv targeting digoxigenin and chelating Eu (red). Liquid phase labeling assured uniform access of probes and antibodies to the targets, which led to uniform labeling. Attachment of chromosome to the substrate after labeling assured a very clean background and good signal to noise ratio. The chromosome was imaged upon multi-photon excitation by the tunable, pulsed laser.

### DETAILED DESCRIPTION

According to the embodiments of the disclosure, death tags that target cells undergoing apoptosis, necrotic cells, other dead or dying cells or circulating free DNA released from said cells are provided herein. The death tags may be used with in vivo or ex vivo methods to detect dead or dying cells, to detect intracellular fragments of dying or decaying cells, or to detect circulating free DNA released from said cells in a physiologic fluid. The death tags and methods for their use described herein may be directed to any suitable application including, but are not limited to, evaluating effectiveness of a cancer therapy that may result in therapeutic or iatrogenic cell death, and determining the extent of injury to a tissue resulting from a pathologic condition (e.g., myocardial infarction or stroke) or traumatic injury (e.g., traffic accidents or improvised explosive device explosions) as discussed further below.

In some embodiments, death tags and the methods for their use as described herein may be used to determine or evaluate the effectiveness of one or more toxic treatments or therapies. Chemotherapeutics, radioactive compounds and other systemic toxic therapies affect both healthy and cancerous cells. Because some cancer cells are resistant to these treatments or therapies, it is important to discover whether any therapeutic effects of the treatment or therapy are seen or occur as soon as possible, so a patient is not exposed to the toxic effects of such treatments or therapies without an associated benefit of cancer cell death. Ineffective therapies should be stopped, adjusted, or replaced with an alternative treatment. The methods described herein provide a swifter and timelier approach for adjusting to ineffective treatments than measurement of tumor size.

In some embodiments, a death tag targets one or more molecules on and/or in cells that are dead or are about to die (e.g., dead cancer cells, dead cardiac cells, and dead stem cells). In other embodiments, methods for designing and manufacturing genetically and/or chemically engineered death tags, and methods for their use are provided herein. According to the embodiments of the disclosure, the death tags have a high qualitative and/or quantitative specificity toward markers on dead cancer cells in vivo or in vitro, a high binding affinity toward dead cell markers to remain bound to the markers for a long period of time, and a reporter binding domain, such as a metal or superparamagnetic nanoparticle for detecting the presence of the death tag. The death tags should also be non-toxic and bio-compatible, so as to not cause any side effects or create any risks of inflicting harm to a patient.

The death tags described herein may be used to detect markers of cell death at any point during the process of cell death and by any manner of death (e.g., apoptosis, necrosis, or injury). For example, a marker of cell death may be an apoptotic marker that is present at the beginning of the apoptotic process or may be an intracellular marker that may only be assessed once a cell's membrane is compromised and permeable, which occurs at the end of most cell death processes, once death is inevitable. Some common chemotherapeutics (e.g., Dexamethasone, Cisplatin) induce apoptosis, therefore, detection of the initiation of cell death and/or ultimate cell death may be used to determine the efficacy of such treatments.

During the apoptosis process, the intracellular contents of dying or decaying cells are retained within cell membranes, but the cell surface receptors undergo changes. These changes occur soon after treatment (minutes to hours) with pro-apoptotic therapies. Loss of membrane integrity and membrane permeability occurs within a similar time frame. Therefore, in some embodiments, the death tags as described herein may be used to detect the initiation of or ultimate cell death as a result of any cause by targeting apoptotic markers on the cell or intracellular contents of dying or decaying cells. In one embodiment, the death tags may be used to detect cancer cell death in response to one or more cancer therapies. In one embodiment, a method for determination of the effectiveness of a delivered cancer therapy is provided that includes administering an effective dose of a death tag to a subject, exposing the subject to an imaging technique, and determining that the cancer treatment is effective when the imaging technique detects the presence of the death tag.

In other embodiments, death tags may be used to detect the extent or existence of cell death as a result of a pathological condition (e.g., myocardial infarction, stroke, hypoxia, ischemia, neoplasms, atrophy of muscles, spontaneous cancer cell death, Parkinson's disease, Alzheimer's disease or other conditions) or traumatic injury or other damage caused by physical or environmental trauma (e.g., frostbite, musculoskeletal injuries, burns, whiplash, brain injury, traffic accidents, or improvised explosive device (IED) explosions).

The methods described herein allow for a prompt evaluation or determination of the effectiveness of a delivered cancer therapy or an immediate or almost immediate evaluation of the lethal injuries to tissues and organs resulting from any cause as described above. Death tags that may be used with the methods described herein are further discussed below.

In some embodiments, the death tags may target cell death markers present at early stages of apoptosis and/or necrosis. Processes leading to cell death involve a characteristic reorganization of biomolecules and macromolecular clusters, which become markers of cell death. Early onset of apoptosis is characterized by formation of membrane “blebbing” and involves flipping of phosphatidyl serine (PS), normally found on the cytosolic side of the membrane, to the external leaflet of cell membranes. The externalization of PS can be detected by annexin, a 35 kDa molecule that binds PS with high efficiency, modified with a suitable reporter. In some embodiments, annexin may be directly labeled with fluorochromes or radioactive isotopes (e.g., Tc99m), or with superparamagnetic ions, or with heavy atoms. However, in accordance with certain embodiments of the disclosure, death tags, such as those described herein, may alternatively be used in a safer, non-radioactive method of detecting early stages of apoptosis and cell death. For example, in one embodiment, a cell death tag may target phosphatidyl serine including an antiPS scFv guided death tag (see Table 1).

Cells that have been weakened by the initiation of apoptosis may recover. Apoptosis may be reversed by natural and/or therapeutic apoptotic signaling pathway blockers. Therefore, detection of markers related to the start of apoptosis or necrotic death may not ensure that the cells will ultimately die, how long it will take for death to occur, or whether the cells will recover. Thus, an affirmative indication of cancer therapy or therapeutic efficacy should be measured by the final signs of cell death. Such signs include, but are not limited to, (i) deterioration of the cell's membranes, (ii) providing access for extracellular markers to a cell's cellular organelles (e.g., intermediate filaments characteristic for a particular cancerous tissue origin), genome, nuclear proteins (e.g., DNA, histones), and other intracellular molecules within the cytoplasmic and nuclei interiors, and (iii) shedding or releasing intracellular molecular content into the physiological or pathological fluids.

After a period of time (within approximately minutes to months), genomic DNA, cell biomarkers, and intracellular molecules are shed or released into the circulation. Thus, upon cell death, some cell molecules, including, but not limited to, fragments of genomic DNA of these cells, appear in the blood or lymph circulation in the form of dsDNA or ssDNA, known as circulating free DNA (cfDNA). cfDNA is found in physiological fluids and is present during early stages of cancer and levels of cfDNA increase at advanced stages of cancer, after surgery, or due to effective therapy, indicating cancer cell death and decay. Necrosis may be induced by other methods of therapy (e.g., IR thermal therapy) or be an end result of apoptosis. In necrotic cells, membrane integrity is compromised, and the intracellular molecules may spill into the physiological fluids, (i.e., interstitial fluid, blood, and lymph). These “spilled” molecules may be used as diagnostic or prognostic markers or targets for the methods described herein. Further, the compromised membrane integrity provides intracellular access to death tags for binding intracellular death marker targets. Macrophages also expel fragments of catabolized DNA. Thus, methods for detecting cell death in vitro, ex vivo, or in vivo are provided to provide a way for clinicians to determine whether a particular treatment or therapy is effective in ultimately killing cancer cells. Such a method provides a more immediate and specific measurement of cancer cell death than evaluation based upon measurement of tumor size, thereby reducing deleterious effects to healthy cells when a treatment is not effective or continuing treatment if it is effective.

In the final stages of the cell death, the integrity of cellular membrane is compromised and becomes freely permeable. This is followed by decay of cellular organelles and molecules. Thus, in some embodiments, the death tags described herein can access the intracellular portion of dying or dead cells and may be used to assess the extent of damage to an organ resulting from brain stroke, myocardial infarction (heart attack), pancreatic infarction, tissues crushed in a traffic accident or due to an improvised explosive device (IED) explosion and other injury to organs and cells resulting in cell death. Further, the death tags are permeable to the endothelium allowing them to detect cell death prior to vascularization or breaking the endothelial barrier in blood brain barrier, blood tumor barrier, blood placenta barrier, etc. Therefore, the death tags may be used in an emergency setting as tools to determine the extent of trauma. For example, loss of membrane integrity as a result of cardiac muscle necrosis exposes the intracellular portion of cardiomyocytes to reveal cardiac myosin (CM). This event makes exposed CM a marker of cell death. For example, a broken membrane of cardiac cells allows a 155 kDa anti-Cardiac-Myosin IgG to enter the cell and can be shown in confocal. Modification of antiCM with radioactive compounds (e.g., Tc99m) (Khaw et al 1980) led to development of a heart muscle injury tag frequently used in nuclear medicine departments for evaluation of the extent of the cardiac muscle injury resulting from heart attack. However, like other radioactive substances, there are drawbacks associated with radioactive antiCM and other radioactive substances. Furthermore, CM is often released from the overworked or injured muscles to the circulation. In these cases CM may give false positive results and saturate the probe. CM is also so unstable that may not be detected if too much time since the injury has elapsed. The death tags and their uses described further below are advantageous for use in a clinical setting.

At least three reliable and stable markers of death were identified: DNA (including dsDNA and ssDNA), lamins, and histones. These and other biomolecules are sealed within a living cell's cellular membrane and nuclear envelope. Once membrane integrity has been compromised and death is inevitable, these protections are no longer intact, but the intracellular biomolecules that are stable markers of death remain confined within cells. Thus, detection of these biomolecules (markers of cell death) may be used as a reliable sign of non-reversible cells death. Design, manufacturing, and uses of death tags that target these biomolecules, are provided herein.

The death tags described herein may comprise multiple domains including, but not limited to, a death marker binding domain, a reporter component and a reporter binding domain. The death tag domains may be associated with each other by any suitable method of conjugation or connection (or association), known in the art. According to some embodiments, the death tag domains may be connected using known methods of linking proteins, peptides, antibodies and functional fragments thereof, metals, atoms and molecules. In one aspect, the domains may be designed with overlapping complementary strands so that they may be joined together. In one aspect, the death tag domains are joined by site-specific conjugation using a suitable linkage or bond. In another aspect, the death tag domains may be joined by a bifunctional linker, the design of which would be known by one skilled in the art. Site-specific conjugation is more likely to preserve the binding activity of an antibody or functional antibody fragment. Alternatively, other linkages or bonds used to connect the death tag domains may include, but is not limited to, a covalent bond, a non-covalent bond, a chemical bond, an electrostatic bond, an intermolecular force, an ionic bond, a hydrogen bond, van der Waal forces, a dipole-dipole interaction, metallic bonds, a sulfide linkage, a hydrazone linkage, a hydrazine linkage, an ester linkage, an amido linkage, and amino linkage, an imino linkage, a thiosemicabazone linkage, a semicarbazone linkage, an oxime linkage and a carbon-carbon linkage. In another aspect the domains may be fused-in-frame, the DNA coding sequences by overlap extension, or may otherwise be formed by a single recombinant protein.

A death marker binding domain that may be used in accordance with the disclosure may be any suitable substance that can target a molecule that is indicative of cell death. The substance may be a natural ligand or antibody; or a synthetic molecule capable of targeting a selected death marker. In one embodiment, the death marker binding domain may be an antibody or functional fragment thereof. An antibody or functional antibody fragment thereof refers to an immunoglobulin (Ig) molecule that specifically binds to, or is immunologically reactive with a particular target antigen, and includes both polyclonal and monoclonal antibodies. The term antibody includes genetically engineered or otherwise modified forms of immunoglobulins functional fragments thereof, such as chimeric antibodies, humanized antibodies, heteroconjugate antibodies (e.g., bispecific antibodies, diabodies, triabodies, tetrabodies, affibodies and minibodies). The term functional antibody fragment includes antigen binding fragments of antibodies including, but not limited to, Fab′ fragments, F(ab′)2 fragments, Fab fragments, Fv fragments, rIgG fragments, single chain variable fragments (scFv), single domain variable fragments (sdFv), complementarity-determining region (CDR) fragments, specificity-determining residue (SDR) fragments, complementary domains (CDs) and their fragments. scFv antibody fragments in which the variable domains of the heavy chain and of the light chain of a traditional two chain antibody have been joined to form one chain by genetic engineering, synthesis, combinatorial chemistry, or other suitable methods. sdFv antibody fragments are single peptide molecules, which are genetically engineered to contain a single domain targeting an epitope.

While any antibody or functional fragment thereof may be suitable for use as a death marker binding domain, a preferred embodiment is an scFv, sdFv, SDR, CDR or other small antibody functional fragment or complementary molecule, which is capable of reducing steric hindrance and increasing sensitivity and specificity as described in Malecki et al., 2002, which is incorporated herein in its entirety as if fully set forth herein. Other small substances may also be suitable for use as a death marker binding domain, including, but not limited to, a nucleic acid, an aptamer, a small molecule, a peptide, a protein, a fusion protein, a chimeric protein, an affibody, or a peptibody. An scFv, sdFv or other molecule derived from a natural antibody or a biomolecule generated by in vitro evolution or synthesized in vitro or modified using their fragments may be used in accordance with the embodiments described herein.

According to some embodiments of the disclosure, a death marker binding domain may target DNA (e.g., dsDNA or ssDNA), histones, lamins or phosphatidyl serine (PS) or any other suitable marker found in dead cells, their fragments, and their integers. In some embodiments, the death marker binding domain may include, but is not limited to the cDNA sequences, consensus codons, mRNA and Fv amino acid sequences found in Table 1.

TABLE 1
Sequences of death marker binding domains for DNA, histones,
lamins and PS

| | | SEQ |
|---|---|---|
| | | ID |
| Target | Sequence | NO: |
| antiDNA Fv | GAAGTGCAGCTGCTGGAAAGCGGCGGCGGCCTGGTGCAGCCGGGCGGCAG | 1 |
| Heavy Chain | CCTGCGCCTGAGCTGCGCGGCGAGCGGCTTTACCTTTAGCAGCTATGCGAT | |
| (cDNA) | GAGCTGGGTGCGCCAGGCGCCGGGCAAAGGCCTGGAATGGGTGAGCGCGA | |
| | TTAGCGGCAGCGGCGGCAGCACCTATTATGCGGATAGCGTGAAAGGCCGCT | |
| | TTACCATTAGCCGCGATAACAGCAAAAACACCCTGTATCTGCAGATGAACAGC | |
| | CTGCGCGCGGAAGATACCGCGGTGTATTATTGCGCGAAAGGCCAGGTGCTG | |
| | TATTATGGCAGCGGCAGCTATCATTGGTTTGATCCGTGGGGCCAGGGCACCC | |
| | TGGTGACCGTGAGCAGC | |
| antiDNA Fv | GARGTNCARYTNYTNGARWSNGGNGGNGGNYTNGTNCARCCNGGNGGNWS | 2 |
| Heavy Chain | NYTNMGNYTNWSNTGYGCNGCNWSNGGNTTYACNTTYWSNWSNTAYGCNAT | |
| (consensus | GWSNTGGGTNMGNCARGCNCCNGGNAARGGNYTNGARTGGGTNWSNGCNA | |
| codon)* | THWSNGGNWSNGGNGGNWSNACNTAYTAYGCNGAYWSNGTNAARGGNMGN | |
| | TTYACNATHWSNMGNGAYAAYWSNAARAAYACNYTNTAYYTNCARATGAAYW | |
| | SNYTNMGNGCNGARGAYACNGCNGTNTAYTAYTGYGCNAARGGNCARGTNYT | |
| | NTAYTAYGGNWSNGGNWSNTAYCAYTGGTTYGAYCCNTGGGGNCARGGNAC | |
| | NYTNGTNACNGTNWSNWSN | |
| antiDNA Fv | GAAGUGCAGCUGCUGGAAAGCGGCGGCGGCCUGGUGCAGCCGGGCGGCA | 3 |
| Heavy Chain | GCCUGCGCCUGAGCUGCGCGGCGAGCGGCUUUACCUUUAGCAGCUAUGC | |
| (mRNA) | GAUGAGCUGGGUGCGCCAGGCGCCGGGCAAAGGCCUGGAAUGGGUGAGC | |
| | GCGAUUAGCGGCAGCGGCGGCAGCACCUAUUAUGCGGAUAGCGUGAAAGG | |
| | CCGCUUUACCAUUAGCCGCGAUAACAGCAAAAACACCCUGUAUCUGCAGAU | |
| | GAACAGCCUGCGCGCGGAAGAUACCGCGGUGUAUUAUUGCGCGAAAGGCC | |
| | AGGUGCUGUAUUAUGGCAGCGGCAGCUAUCAUUGGUUUGAUCCGUGGGGC | |
| | CAGGGCACCCUGGUGACCGUGAGCAGC | |
| antiDNA Fv | EVQLLESGGGLVQPGGSLRLSCAASGFTFSSYAMSWVRQAPGKGLEWVSAISG | 4 |
| Heavy Chain | SGGSTYYADSVKGRFTISRDNSKNTLYLQMNSLRAEDTAVYYCAKGQVLYYGSG | |
| (amino acid) | SYHWFDPWGQGTLVTVSS | |
| antiDNA Fv | GATATTCAGATGACCCAGAGCCCGAGCAGCCTGAGCGCGAGCGTGGGCGAT | 5 |
| Light Chain | CGCGTGACCATTACCTGCCGCGCGAGCCAGGGCATTCGCAACGATCTGGGC | |
| (cDNA) | TGGTATCAGCAGAAACCGGGCAAAGCGCCGAAACGCCTGATTTATGCGGCG | |
| | AGCAGCCTGGAAAGCGGCGTGCCGAGCCGCTTTAGCGGCAGCGGCAGCGG | |
| | CACCGAATTTACCCTGACCATTAGCAGCCTGCAGCCGGAAGATTTTGCGACC | |
| | TATTATTGCCTGCAGCATAACAGCTATCCGCTGACCTTTGGCGGCGGCACCA | |
| | AAGTGGAAATTAAACGCACC | |
| antiDNA Fv | GAYATHCARATGACNCARWSNCCNWSNWSNYTNWSNGCNWSNGTNGGNGA | 6 |
| Light Chain | YMGNGTNACNATHACNTGYMGNGCNWSNCARGGNATHMGNAAYGAYYTNGG | |
| (consensus | NTGGTAYCARCARAARCCNGGNAARGCNCCNAARMGNYTNATHTAYGCNGC | |
| codon)* | NWSNWSNYTNGARWSNGGNGTNCCNWSNMGNTTYWSNGGNWSNGGNWSN | |
| | GGNACNGARTTYACNYTNACNATHWSNWSNYTNCARCCNGARGAYTTYGCNA | |
| | CNTAYTAYTGYYTNCARCAYAAYWSNTAYCCNYTNACNTTYGGNGGNGGNAC | |
| | NAARGTNGARATHAARMGNACN | |
| antiDNA Fv | AUAUUCAGAUGACCCAGAGCCCGAGCAGCCUGAGCGCGAGCGUGGGCGAU | 7 |
| Light Chain | CGCGUGACCAUUACCUGCCGCGCGAGCCAGGGCAUUCGCAACGAUCUGGG | |
| (mRNA) | CUGGUAUCAGCAGAAACCGGGCAAAGCGCCGAAACGCCUGAUUUAUGCGG | |
| | CGAGCAGCCUGGAAAGCGGCGUGCCGAGCCGCUUUAGCGGCAGCGGCAG | |
| | CGGCACCGAAUUUACCCUGACCAUUAGCAGCCUGCAGCCGGAAGAUUUUG | |
| | CGACCUAUUAUUGCCUGCAGCAUAACAGCUAUCCGCUGACCUUUGGCGGC | |
| | GGCACCAAAGUGGAAAUUAAACGCACC | |
| antiDNA Fv | DIQMTQSPSSLSASVGDRVTITCRASQGIRNDLGWYQQKPGKAPKRLIYAASSLE | 8 |
| Light Chain | SGVPSRFSGSGSGTEFTLTISSLQPEDFATYYCLQHNSYPLTFGGGTKVEIKRT | |
| (amino acid) | | |
| antiHistone | GATATTCAGATGACCCAGAGCCCGAGCAGCCTGAGCGCGAGCGTGGGCGAT | 9 |
| Light Chain | CGCGTGACCATTAGCTGCACCGGCCCGAGCAGCCCGGTGGGCGGCTATAAA | |
| (cDNA) | CCGATTAGCTGGTATCATCAGCATCCGGGCACCGCGCCGAAACTGATGATTT | |
| | ATATTCGCGGCATGCAGCGCAGCGGCGTGCCGGATCGCTTTAGCGGCAGCA | |
| | AAAGCGGCAACACCGCGAGCCTGACCATTAGCGGCCTGCGCGCGATGGATG | |
| | AAGCGGATTATTATTGCGCGCAGTATGATGAACTGCCGTATACCTTTGGCCAG | |
| | GGCACCAAACTGGAAGTGAAACGC | |
| antiHistone | GAYATHCARATGACNCARWSNCCNWSNWSNYTNWSNGCNWSNGTNGGNGA | 10 |
| Light Chain | YMGNGTNACNATHWSNTGYACNGGNCCNWSNWSNCCNGTNGGNGGNTAYA | |
| (consensus | ARCCNATHWSNTGGTAYCAYCARCAYCCNGGNACNGCNCCNAARYTNATGAT | |
| codon)* | HTAYATHMGNGGNATGCARMGNWSNGGNGTNCCNGAYMGNTTYWSNGGNW | |
| | SNAARWSNGGNAAYACNGCNWSNYTNACNATHWSNGGNYTNMGNGCNATG | |
| | GAYGARGCNGAYTAYTAYTGYGCNCARTAYGAYGARYTNCCNTAYACNTTYG | |
| | GNCARGGNACNAARYTNGARGTNAARMGN | |
| antiHistone | GAUAUUCAGAUGACCCAGAGCCCGAGCAGCCUGAGCGCGAGCGUGGGCGA | 11 |
| Light Chain | UCGCGUGACCAUUAGCUGCACCGGCCCGAGCAGCCCGGUGGGCGGCUAUA | |
| (mRNA) | AACCGAUUAGCUGGUAUCAUCAGCAUCCGGGCACCGCGCCGAAACUGAUG | |
| | AUUUAUAUUCGCGGCAUGCAGCGCAGCGGCGUGCCGGAUCGCUUUAGCGG | |
| | CAGCAAAAGCGGCAACACCGCGAGCCUGACCAUUAGCGGCCUGCGCGCGA | |
| | UGGAUGAAGCGGAUUAUUAUUGCGCGCAGUAUGAUGAACUGCCGUAUACC | |
| | UUUGGCCAGGGCACCAAACUGGAAGUGAAACGC | |
| antiHistone | DIQMTQSPSSLSASVGDRVTISCTGPSSPVGGYKPISWYHQHPGTAPKLMIYIRG | 12 |
| Light Chain | MQRSGVPDRFSGSKSGNTASLTISGLRAMDEADYYCAQYDELPYTFGQGTKLEV | |
| (amino acid) | KR | |
| antiHistone | CAGGTGATGCAGCTGGTGGAAAGCGGCGGCGGCCTGGTGCAGCCGGGCCG | 13 |
| Heavy Chain | CAGCCTGCGCCTGAGCTGCGCGGCGAGCGGCTTTACCTTTAACGATTATCCG | |
| (cDNA) | CTGCATTGGGTGCGCCAGCCGCCGGGCAAAGGCCTGGAATGGAGCAGCGG | |
| | CATTAGCTGGAACAGCGGCAGCATTGGCTATGCGGATAGCGTGAAAGGCCG | |
| | CTTTACCATTAGCCGCGATAACGCGAAAAACAGCCTGTATCTGCAGATGAACA | |
| | GCCTGCGCGCGGAAGATACCGCGCTGTATTATTGCGCGAAAGGCCCGCCGG | |
| | GCTATTATGATAGCAGCGAACCGAGCGATTGGGGCCAGGGCCATGGCCATC | |
| | TGGTGACCGTGAGCAGC | |
| antiHistone | CARGTNATGCARYTNGTNGARWSNGGNGGNGGNYTNGTNCARCCNGGNMG | 14 |
| Heavy Chain | NWSNYTNMGNYTNWSNTGYGCNGCNWSNGGNTTYACNTTYAAYGAYTAYCC | |
| (consensus | NYTNCAYTGGGTNMGNCARCCNCCNGGNAARGGNYTNGARTGGWSNWSNG | |
| codon)* | GNATHWSNTGGAAYWSNGGNWSNATHGGNTAYGCNGAYWSNGTNAARGGN | |
| | MGNTTYACNATHWSNMGNGAYAAYGCNAARAAYWSNYTNTAYYTNCARATGA | |
| | AYWSNYTNMGNGCNGARGAYACNGCNYTNTAYTAYTGYGCNAARGGNCCNC | |
| | CNGGNTAYTAYGAYWSNWSNGARCCNWSNGAYTGGGGNCARGGNCAYGGN | |
| | CAYYTNGTNACNGTNWSNWSN | |
| antiHistone | CAGGUGAUGCAGCUGGUGGAAAGCGGCGGCGGCCUGGUGCAGCCGGGCC | 15 |
| Heavy Chain | GCAGCCUGCGCCUGAGCUGCGCGGCGAGCGGCUUUACCUUUAACGAUUAU | |
| (mRNA) | CCGCUGCAUUGGGUGCGCCAGCCGCCGGGCAAAGGCCUGGAAUGGAGCA | |
| | GCGGCAUUAGCUGGAACAGCGGCAGCAUUGGCUAUGCGGAUAGCGUGAAA | |
| | GGCCGCUUUACCAUUAGCCGCGAUAACGCGAAAAACAGCCUGUAUCUGCA | |
| | GAUGAACAGCCUGCGCGCGGAAGAUACCGCGCUGUAUUAUUGCGCGAAAG | |
| | GCCCGCCGGGCUAUUAUGAUAGCAGCGAACCGAGCGAUUGGGGCCAGGGC | |
| | CAUGGCCAUCUGGUGACCGUGAGCAGC | |
| antiHistone | QVMQLVESGGGLVQPGRSLRLSCAASGFTFNDYPLHWVRQPPGKGLEWSSGIS | 16 |
| Heavy Chain | WNSGSIGYADSVKGRFTISRDNAKNSLYLQMNSLRAEDTALYYCAKGPPGYYDS | |
| (amino acid) | SEPSDWGQGHGHLVTVSS | |
| antiLamin | GATATTCAGATGACCCAGAGCCCGAGCAGCCTGAGCGCGAGCGTGGGCGAT | 17 |
| Light Chain | CGCGTGACCATTACCTGCAGCGGCGATAAACTGGGCGATAAATATGCGTGCT | |
| (cDNA) | GGTATCAGCAGAAACCGGGCCAGAGCCCGGTGCTGGTGATTTATCAGGATA | |
| | GCAAACGCCCGAGCGGCATTCCGGAACGCTTTAGCGGCAGCAACAGCGGCA | |
| | ACACCGCGACCCTGACCATTAGCGGCACCCAGGCGATGGATGAAGCGGATT | |
| | ATTATTGCAACGCGTGGGATAGCAGCACCTATGTGGTGTTTGGCGGCGGCAC | |
| | CAAACTGACCGTGCTGGGCCAGCCG | |
| antiLamin | GAYATHCARATGACNCARWSNCCNWSNWSNYTNWSNGCNWSNGTNGGNGA | 18 |
| Light Chain | YMGNGTNACNATHACNTGYWSNGGNGAYAARYTNGGNGAYAARTAYGCNTG | |
| (consensus | YTGGTAYCARCARAARCCNGGNCARWSNCCNGTNYTNGTNATHTAYCARGAY | |
| codon)* | WSNAARMGNCCNWSNGGNATHCCNGARMGNTTYWSNGGNWSNAAYWSNG | |
| | GNAAYACNGCNACNYTNACNATHWSNGGNACNCARGCNATGGAYGARGCNG | |
| | AYTAYTAYTGYAAYGCNTGGGAYWSNWSNACNTAYGTNGTNTTYGGNGGNG | |
| | GNACNAARYTNACNGTNYTNGGNCARCCN | |
| antiLamin | GAUAUUCAGAUGACCCAGAGCCCGAGCAGCCUGAGCGCGAGCGUGGGCGA | 19 |
| Light Chain | UCGCGUGACCAUUACCUGCAGCGGCGAUAAACUGGGCGAUAAAUAUGCGU | |
| (mRNA) | GCUGGUAUCAGCAGAAACCGGGCCAGAGCCCGGUGCUGGUGAUUUAUCAG | |
| | GAUAGCAAACGCCCGAGCGGCAUUCCGGAACGCUUUAGCGGCAGCAACAG | |
| | CGGCAACACCGCGACCCUGACCAUUAGCGGCACCCAGGCGAUGGAUGAAG | |
| | CGGAUUAUUAUUGCAACGCGUGGGAUAGCAGCACCUAUGUGGUGUUUGGC | |
| | GGCGGCACCAAACUGACCGUGCUGGGCCAGCCG | |
| antiLamin | DIQMTQSPSSLSASVGDRVTITCSGDKLGDKYACWYQQKPGQSPVLVIYQDSKR | 20 |
| Light Chain | PSGIPERFSGSNSGNTATLTISGTQAMDEADYYCNAWDSSTYVVFGGGTKLTVL | |
| (amino acid) | GQP | |
| antiLamin | GAAGTGCAGCTGGTGGAAAGCGGCGGCGGCCTGGTGCAGCCGGGCGGCAG | 21 |
| Heavy Chain | CCTGCGCCTGAGCTGCGCGGCGAGCGGCTTTACCTTTAGCAGCTATAGCATG | |
| (cDNA) | AGCTGGGTGCGCCAGGCGCCGGGCAAAGGCCTGGAATGGATTAGCTATATT | |
| | AGCAGCAGCAGCAGCACCATTTATTATGCGGATAGCGTGAAAGGCCGCTTTA | |
| | CCATTAGCCGCGATAACGCGAAAAACAGCCTGTATCTGCAGATGAACAGCCT | |
| | GCGCGCGGAAGATACCGCGGTGTATTATTGCGCGCGCAGCCGCAACTATGA | |
| | TAGCAGCGGCTATTATAGCCATTATTTTGATTTTTGGGGCCAGGGCACCATGG | |
| | TGACCGTGAGCAGC | |
| antiLamin | GARGTNCARYTNGTNGARWSNGGNGGNGGNYTNGTNCARCCNGGNGGNWS | 22 |
| Heavy Chain | NYTNMGNYTNWSNTGYGCNGCNWSNGGNTTYACNTTYWSNWSNTAYWSNAT | |
| (consensus | GWSNTGGGTNMGNCARGCNCCNGGNAARGGNYTNGARTGGATHWSNTAYA | |
| codon)* | THWSNWSNWSNWSNWSNACNATHTAYTAYGCNGAYWSNGTNAARGGNMGN | |
| | TTYACNATHWSNMGNGAYAAYGCNAARAAYWSNYTNTAYYTNCARATGAAYW | |
| | SNYTNMGNGCNGARGAYACNGCNGTNTAYTAYTGYGCNMGNWSNMGNAAYT | |
| | AYGAYWSNWSNGGNTAYTAYWSNCAYTAYTTYGAYTTYTGGGGNCARGGNA | |
| | CNATGGTNACNGTNWSNWSN | |
| antiLamin | GAAGUGCAGCUGGUGGAAAGCGGCGGCGGCCUGGUGCAGCCGGGCGGCA | 23 |
| Heavy Chain | GCCUGCGCCUGAGCUGCGCGGCGAGCGGCUUUACCUUUAGCAGCUAUAGC | |
| (mRNA) | AUGAGCUGGGUGCGCCAGGCGCCGGGCAAAGGCCUGGAAUGGAUUAGCUA | |
| | UAUUAGCAGCAGCAGCAGCACCAUUUAUUAUGCGGAUAGCGUGAAAGGCC | |
| | GCUUUACCAUUAGCCGCGAUAACGCGAAAAACAGCCUGUAUCUGCAGAUGA | |
| | ACAGCCUGCGCGCGGAAGAUACCGCGGUGUAUUAUUGCGCGCGCAGCCGC | |
| | AACUAUGAUAGCAGCGGCUAUUAUAGCCAUUAUUUUGAUUUUUGGGGCCA | |
| | GGGCACCAUGGUGACCGUGAGCAGC | |
| antiLamin | EVQLVESGGGLVQPGGSLRLSCAASGFTFSSYSMSWVRQAPGKGLEWISYISSS | 24 |
| Heavy Chain | SSTIYYADSVKGRFTISRDNAKNSLYLQMNSLRAEDTAVYYCARSRNYDSSGYYS | |
| (amino acid) | HYFDFWGQGTMVTVSS | |
| antiPS | GAAGTGCAGCTGCTGGAAAGCGGCGGCGGCCTGGTGCAGCCGGGCGGCAG | 25 |
| Heavy Chain | CCTGCGCCTGAGCTGCGCGGCGAGCGGCTTTACCTTTAGCAGCTATGCGAT | |
| (cDNA) | GAGCTGGGTGCGCCAGGCGCCGGGCAAAGGCCTGGAATGGGTGAGCGCGA | |
| | TTAGCGGCAGCGGCGGCAGCACCTATTATGCGGATAGCGTGAAAGGCCGCT | |
| | TTACCATTAGCCGCGATAACAGCAAAAACACCCTGTATCTGCAGATGAACAGC | |
| | CTGCGCGCGGAAGATACCGCGGTGTATTATTGCGCGAAAGATGGCGAATATG | |
| | AAGCGGGCATTGATTATTGGTTTGATCCGTGGGGCCAGGGCACCCTG | |
| antiPS | GARGTNCARYTNYTNGARWSNGGNGGNGGNYTNGTNCARCCNGGNGGNWS | 26 |
| Heavy Chain | NYTNMGNYTNWSNTGYGCNGCNWSNGGNTTYACNTTYWSNWSNTAYGCNAT | |
| (consensus | GWSNTGGGTNMGNCARGCNCCNGGNAARGGNYTNGARTGGGTNWSNGCNA | |
| codon)* | THWSNGGNWSNGGNGGNWSNACNTAYTAYGCNGAYWSNGTNAARGGNMGN | |
| | TTYACNATHWSNMGNGAYAAYWSNAARAAYACNYTNTAYYTNCARATGAAYW | |
| | SNYTNMGNGCNGARGAYACNGCNGTNTAYTAYTGYGCNAARGAYGGNGART | |
| | AYGARGCNGGNATHGAYTAYTGGTTYGAYCCNTGGGGNCARGGNACNYTN | |
| antiPS | GAAGUGCAGCUGCUGGAAAGCGGCGGCGGCCUGGUGCAGCCGGGCGGCA | 27 |
| Heavy Chain | GCCUGCGCCUGAGCUGCGCGGCGAGCGGCUUUACCUUUAGCAGCUAUGC | |
| (mRNA) | GAUGAGCUGGGUGCGCCAGGCGCCGGGCAAAGGCCUGGAAUGGGUGAGC | |
| | GCGAUUAGCGGCAGCGGCGGCAGCACCUAUUAUGCGGAUAGCGUGAAAGG | |
| | CCGCUUUACCAUUAGCCGCGAUAACAGCAAAAACACCCUGUAUCUGCAGAU | |
| | GAACAGCCUGCGCGCGGAAGAUACCGCGGUGUAUUAUUGCGCGAAAGAUG | |
| | GCGAAUAUGAAGCGGGCAUUGAUUAUUGGUUUGAUCCGUGGGGCCAGGGC | |
| | ACCCUG | |
| antiPS | EVQLLESGGGLVQPGGSLRLSCAASGFTFSSYAMSWVRQAPGKGLEWVSAISG | 28 |
| Heavy Chain | SGGSTYYADSVKGRFTISRDNSKNTLYLQMNSLRAEDTAVYYCAKDGEYEAGID | |
| (amino acid) | YWFDPWGQGTL | |
| antiPS | GATATTCAGATGACCCAGAGCCCGAGCAGCCTGAGCGCGAGCGTGGGCGAT | 29 |
| Light Chain | CGCGTGACCATTACCTGCCGCAGCAGCCAGGATATTAACAAATATATTGGCT | |
| (cDNA) | GGTATCAGCAGAAACCGGGCAAAGCGCCGAAACTGCTGATTCATTATACCAG | |
| | CACCCTGCAGCCGGGCGTGCCGAGCCGCTTTAGCGGCAGCGGCAGCGGCA | |
| | CCGATTTTACCCTGACCATTAGCAGCCTGCAGCCGGAAGATTTTGCGACCTAT | |
| | TTTTGCCTGAACTATGATAACCTGTATAGCTTTGGCGGCGGCACCAAAGTGGA | |
| | AATTAAACGC | |
| antiPS | GAYATHCARATGACNCARWSNCCNWSNWSNYTNWSNGCNWSNGTNGGNGA | 30 |
| Light Chain | YMGNGTNACNATHACNTGYMGNWSNWSNCARGAYATHAAYAARTAYATHGG | |
| (consensus | NTGGTAYCARCARAARCCNGGNAARGCNCCNAARYTNYTNATHCAYTAYACN | |
| codon)* | WSNACNYTNCARCCNGGNGTNCCNWSNMGNTTYWSNGGNWSNGGNWSNG | |
| | GNACNGAYTTYACNYTNACNATHWSNWSNYTNCARCCNGARGAYTTYGCNAC | |
| | NTAYTTYTGYYTNAAYTAYGAYAAYYTNTAYWSNTTYGGNGGNGGNACNAARG | |
| | TNGARATHAARMGN | |
| antiPS | GAUAUUCAGAUGACCCAGAGCCCGAGCAGCCUGAGCGCGAGCGUGGGCGA | 31 |
| Light Chain | UCGCGUGACCAUUACCUGCCGCAGCAGCCAGGAUAUUAACAAAUAUAUUGG | |
| (mRNA) | CUGGUAUCAGCAGAAACCGGGCAAAGCGCCGAAACUGCUGAUUCAUUAUAC | |
| | CAGCACCCUGCAGCCGGGCGUGCCGAGCCGCUUUAGCGGCAGCGGCAGC | |
| | GGCACCGAUUUUACCCUGACCAUUAGCAGCCUGCAGCCGGAAGAUUUUGC | |
| | GACCUAUUUUUGCCUGAACUAUGAUAACCUGUAUAGCUUUGGCGGCGGCA | |
| | CCAAAGUGGAAAUUAAACGC | |
| antiPS | DIQMTQSPSSLSASVGDRVTITCRSSQDINKYIGWYQQKPGKAPKLLIHYTSTLQP | 32 |
| Light Chain | GVPSRFSGSGSGTDFTLTISSLQPEDFATYFCLNYDNLYSFGGGTKVEIKR | |
| (amino acid) | | |

*The consensus codons are degeneracy sequences which follow the standard IUPAC symbols for DNA (R = A or G; Y = C or T; M = A or C; W = A or T; S = C or G; B = C, G or T; D = A, G or T; H = A, C or T; V = A, C or G; and N is any nucleotide (A, C G or T)).

In some embodiments, the death tags may include a reporter component. The use of a reporter component allows for visualization and/or quantification of the death tags via use of diagnostic imaging techniques such as x-ray radiography, computed tomography (CT), magnetic resonance imaging (MRI), Raman, gamma scintigraphy, Raman spectral imaging (RSI), positron emission tomography (PET), single photon emission computed tomography (SPECT), ultrasonography (USG), fluorescence imaging (e.g., fluorometry, fluorescein (FL) or FL-derivative imaging), scintillation, NMR and/or NMR miniscanning and surface plasmon resonance (SPR).

According to some embodiments, a reporter component may be any suitable diagnostic or imaging substance that may be detected by an imaging device or sensor, while being associated with a reporter binding domain and a death marker binding domain. For example, the death tags described herein may be combined with a contrast for use with radiography, computed tomography (CT), magnetic resonance imaging (MRI), ultrasonography (USG), and Raman spectral imaging (RSI) as described below. Alternatively, the death tags may be modified to accept radionuclides for use with nuclear medicine techniques, such as positron emission tomography (PET), single photon emission computed tomography (SPECT) and gamma scintigraphy.

Reporter components that may be used in accordance with the embodiments described herein may include, but are not limited to, metal nanoparticles, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions), or a fluorochrome or a microbubble or a radionuclide.

In one embodiment, the reporter component is a metal nanoparticle. The metal nanoparticles may be formed from a single suitable solid metal or from a combination of two or more suitable metals. In some embodiments, the metal nanoparticle tag may comprise a nanoparticle derived from a noble metal, including, but not limited to, Gold (Au), Platinum (Pt), Palladium (Pd) and Silver (Ag). In other embodiments, the metal nanoparticle may comprise a superparamagnetic metal, including, but not limited to, Europium (Eu), Gadolinium (Gd), Iron (Fe), Nickel (Ni) or Cobalt (Co). In other embodiments, the metal nanoparticle may comprise a nanoparticle derived from a fluorescent metal, including, but not limited to, Europium (Eu) and Terbium (Tb). Some metal nanoparticles can be made as chelated nanoclusters or as core-shell nanoparticles, which have a superparamagnetic, heavy metal or fluorescent metal core that is sealed inside a noble-metal layer (or “core-shell”). Other nanoparticles may be made as a “microbubble” nanoparticle, having a noble metal outer core-shell layer, with a hollow core. In addition, it should be noted that some nanoparticles, for example, quantum dots, may also be suitable for use as a detection agent.

Radioactive substances that may be used as a reporter component in accordance with the embodiments of the disclosure include, but are not limited to, ¹⁸F, ³²P, ³³P, ⁴⁵Ti, ⁴⁷Sc, ⁵²Fe, 59Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y. ⁸⁹Sr, ⁸⁹Zr, ⁹⁴Tc, ⁹⁴Tc, ^{99m}Tc, ^{99m}Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as reporter components in accordance with the embodiments of the disclosure include, but are not limited to, ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, Ru, and Lu.

Contrast agents that may be used as reporter components in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof. Targeted contrast agents that may be used according to the embodiments described herein are described in further detail below.

Bioluminescent and fluorescent compounds or molecules and dyes that may be used as reporter components in accordance with the embodiments of the disclosure include, but are not limited to, fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, fluorescent metals including, but not limited to Eu, Tb, Ru, fluorescent amino acids (e.g., Tyrosine), or combination thereof. According to embodiments described herein, a fluorescent reporter may be used to measure by flow cytometry (FCM) and/or sort cells targeted by the death tags described herein using fluorescent flow cytometry methods known in the art including, but not limited to, fluorescence-activated cell sorting (FACS).

Enzymes that may be used as reporter components in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

According to some embodiments, the death tags described herein include a reporter binding domain to provide a binding site for the reporter compound. The reporter binding domain may be a metal binding domain (MBD), a chelating site or an organic functional group (e.g., amino, carboxyl, thiol or azide groups) or a synthetic chelate (e.g., DTPA or DOTA). For example, when the reporter component is a metal (e.g., a noble metal or superparamagnetic metal) or paramagnetic ion, the death tag may include a metal binding domain. In such case, the reporter component may be reacted with a reagent having a long tail with one or more chelating groups attached to the long tail for binding these ions. The long tail may be a polymer such as a polylysine, polysaccharide, or other derivatized or derivatizable chain having pendant groups to which may be bound to a chelating group for binding the ions. Examples of chelating groups that may be used according to the disclosure include, but are not limited to, ethylenediaminetetraacetic acid (EDTA), EGTA, diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the antibody or functional antibody fragment by a group which enables formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium, gadolinium, iodine, and copper, respectively. In certain embodiments, chelating moieties may be used to attach a PET imaging agent, such as an Al-¹⁸F complex, to a targeting molecule for use in PET analysis.

According to some embodiments, a metal binding domain (MBD) that is part of a death tag described herein may include, but is not limited to, the following sequences, or any functional fragment thereof:

(SEQ ID NO: 33)
(Gly)n-Cys;
(SEQ ID NO: 34)
(Gly-Arg-)n-Cys;
(SEQ ID NO: 35)
(Gly-Lys-)n-CyS;
(SEQ ID NO: 36)
(Gly-Asp-Gly-Arg)n-Cys;
(SEQ ID NO: 37)
(Gly-Glu-Gly-Arg)n-Cys;
(SEQ ID NO: 38)
(Gly-Asp-Gly-Lys)n-Cys;
(SEQ ID NO: 39)
(Gly-Glu-Gly-Lys)n-Cys;
(SEQ ID NO: 40)
MAP16-B; (Glu-Glu-Glu-Glu-Glu)n;
(SEQ ID NO: 41)
(Glu-Glu-Glu-Glu-Glu-Glu)n;
(SEQ ID NO: 42)
(Asp-Asp-Asp-Asp-Asp)n;
(SEQ ID NO: 43)
(Asp-Asp-Asp-Asp-Asp-Asp)n;
(SEQ ID NO: 44)
Phe-His-Cys-Pro-Tyr-Asp-Leu-Cys-His-Ile-Leu;
(SEQ ID NO: 45)
(Gly-Asp-Gly-Arg)n-(His)5,6;
(SEQ ID NO: 46)
(Gly-Glu-Gly-Arg)n-(His)5,6;
(SEQ ID NO: 47)
(Gly-Asp-Gly-Lys)n-(His)5,6;
(SEQ ID NO: 48)
(Gly-Glu-Gly-Lys)n-(His)5,6;
(SEQ ID NO: 49)
(Gly-Arg-)n-(His)5,6;
or
(SEQ ID NO: 50)
(Gly-Lys-v-(His)5,6.

In Vivo Use of Death Tags

In some embodiments, the death tags described herein may be used to detect cell death in response to a therapy or therapeutic regimen. In another embodiment, the death tags may be used to detect the presence or extent of cell death resulting from a pathological condition or disease or a traumatic injury (e.g., after a myocardial infarction, brain stroke, pancreatic infarct, or other pathological condition resulting in direct tissues damage, ischemia, or other causes that can leading to cell death.

Although some tumor cells die spontaneously, the number of spontaneous deaths is negligible as compared to the number of deaths resulting from an effective treatment or therapy. After a population of cells die, their cellular products are eventually released in the blood and lymph circulation due to deterioration of the endothelium or advancing vascularization and the progressing inflammatory response. Currently, evaluation of a therapy's efficacy is determined after the dead cell components are released into the blood and lymph. However, the death tags and the methods for their use as described herein can detect cell death in situ before the dead cells are released in the blood, lymph or other physiological fluids (e.g., peritoneal fluid, serum, plasma, cerebrospinal fluid and urine). Dead cells that are present or accumulate at the site of injury, prior to dilution into an average volume of 5 L of blood, create a stronger signal, which easily reaches the detection threshold of molecular imaging devices. Thus, the embodiments described herein provide methods for detecting dead cells to be before the cellular components become diluted by release into the blood, lymph or physiological fluid. This allows for an early and more sensitive method of detecting cell death.

The methods for detecting cell death as described herein are in vivo cell viability determinations. In cell culture, viability is determined by stains that are impermeable to intact membranes (e.g., trypan blue for light microscopy and propidium iodide for fluorescence microscopy). Cells that resist staining indicate that these cells are viable. However, penetration of cell membranes and staining of cell organelles (e.g., PI stains nucleic acids) is indicative of dead cells. These stains are toxic and therefore not suitable for in vivo or in situ imaging. Therefore, in some embodiments, the death tags may be used in place of or in conjunction with non-toxic stains that have ability to stain the dead cells and be detected in situ, but would not stain viable cells or be toxic to living cells that may or may not be undergoing apoptosis or necrosis.

In some embodiments, methods for use of a targeted contrast composition during a diagnostic imaging technique are provided for localization of dead or dying cells. The methods described herein allow practitioners such as radiologists, oncologists, emergency room physicians and military physicians and staff to detect lethal events using a radiation dose which is much lower than currently used, and the methods allow practitioners to determine or evaluate the effectiveness of administered cancer therapy based upon the rate the cancer cells are dying.

In some embodiments, the methods described herein may be used for determining the extent of and treating injuries obtained in automobile accidents. According to NHTSA, over 30,000 people were killed in traffic accidents in the United States in 2009. Internal bleeding, tissue maceration and organ damage followed by tissue apoptosis and necrosis are often the reasons for long term disabilities and/or death after automobile accidents. The symptoms are often undetected during the physical examinations.

In other embodiments, the methods described herein may be used for determining the extent of and for treating injuries obtained from improvised explosive device (IED) blasts or other explosive device encountered by a soldier deployed in the field. Currently, IEDs are one of the primary causes of casualties in the wars in Iraq and Afghanistan. In 2009, 3366 US soldiers were wounded by IEDs (Vanden 2011). In Afghanistan and Iraq, detection of cell damage and location of the damaged tissue are the first steps towards the medical interventions, which could save lives and prevent severe disabilities of the soldiers.

The markers of cell death allow for the in vitro detection of cell death by detecting the presence and/or the levels of the intracellular molecules in the blood, plasma, serum, lymph, peritoneal fluid, pleural fluid, cerebrospinal fluid or any other physiological fluid as sensors in a point of care (POC) device by minimally trained persons. They also allow for the determination of the location of the damaged cells, tissues, and organs by the spectrum of molecular imaging modalities. Therefore, they allow us instant detection, diagnosis, and targeted therapy of patients.

Furthermore, incorporation of death tags into point of care devices (i.e., treatment or diagnostic devices that are used within close proximity of a patient in the field) for use in the field provides the ability to determine the severity of an injury by untrained soldiers or medics on battle fields or by first response crews on accident sites. For example, such point of care devices may be used to assess whether a wounded soldier or an accident victim has internal injuries, allowing the patient to be treated more effectively in the field, or such information can be relayed to a medical team or hospital to better prepare for receiving the patient.

Therefore, the death tags described herein allow for the determination of therapy effectiveness soon after the onset of the therapy—within minutes to hours—which is much earlier than with the existing methods. This should reduce side effects to the patients, allow for selection of the most effective therapy soon after diagnosis, and would reduce the cost of cancer therapies significantly by not wasting time on ineffective treatment. Further, the death tags may be used in methods for determining the need for a treatment or determining the efficacy of a treatment after injury. For example, the death tags allow for the determination of whether traumatic injuries may require surgery to remove damaged tissue or stop internal bleeding.

In other embodiments, the dead cell molecular imaging techniques described herein may be used for detection and evaluation of a tissue or organ injury resulting from any insult (e.g., frost bites, heat injuries, blunt force trauma, myocardial infarction, stroke, ischemic attack, IED blast, traffic accidents, or other causes of cell damage and death).

As described above, a death tag used for detection and diagnosis of cell death in cancer malignancy may be produced via genetic and chemical engineering of death marker binding domains, which target PS, genomic DNA (e.g., ssDNA or dsDNA), lamins, histones; reporter binding domains and reporter components such as metal nanoparticle tags. In one embodiment, the death tag includes one or more scFv, sdFv, CDR, SDR fragments, as death marker binding domains, a metal binding domain and a metal ionic or nanoparticle reporter component, for example a gold nanoparticle tag. The gold-tagged death tag, or other noble metal-tagged death tag reduces and/or eliminates toxicity and may be used for determining levels of cell death. When used as part of a targeted contrast composition, the gold-tagged death tag may be a safe method for evaluation of therapy with no side effects to healthy tissues. According to some embodiments, the cancer cells labeled with the death tag may be detected with CT with greater sensitivity under significantly lower doses of radiation than currently used. Importantly, these death tags outline the death zones within anatomical topography of the entire body revealed in CT.

In other embodiments, the dying or dead cells labeled with the death tags may also be detected with magnetic resonance imaging (MRI). MRI offers good spatial resolution as compared to other in vivo imaging modalities currently available, and also provides a topographic reference for the location of the death tags within the anatomy of the human body.

Quantitative analysis of each of the death tags, their ratios, time-line of the changes, and total concentration allow physicians to broadcast rational diagnosis, prognosis and plan and modify targeted therapy. Moreover, by determining the location of the death tags, they can serve as targeted radio-sensitizers for delivering radiation therapy with great precision. For example, in some embodiments, targeted delivery of such death tags having noble metal or superparamagnetic nanoparticle tags, can be followed by exposure to CT or MRI respectively, which cause the cancer cells' deaths.

The death tags can be administered in an effective dose to a subject with or without a contrast agent. An effective dose of a death tag with or without a contrast agent for purposes herein is determined by such considerations as are known in the art. For example, an effective amount of the death tag is that amount necessary to deliver a sufficient amount of the death tag such that cells expressing, containing or exposing a cell death marker may be visualized by one or more imaging techniques. Alternatively, an effective amount of the death tag is that amount necessary to deliver a sufficient amount of the death tag to a physiological fluid to remove the death cell marker from the physiological fluid. One of skill in the art can readily determine appropriate single dose sizes for systemic administration based on the size of the patient and the route of administration.

An effective dose of the death tag, with a contrast agent, can be selected according to techniques known to those skilled in the art such that a sufficient contrast enhancing effect is obtained. The targeted contrast agents can be administered by any suitable route depending on the type of procedure and anatomical orientation of the tissue being examined. Suitable administration routes include any administration pathway including, but not limited to, inhalation, enteral, nasal, ophthalmic, oral, parenteral, rectal, intraperitoneal, intrapleural, intratumoral, vaginal, as well as directly into blood, lymph, or cerebrospinal fluid. Parenteral administration refers to a route of administration that is generally associated with injection or catheter, including infraorbital, infusion, intraarterial, intracapsular, intracardiac, intracisternal, intradermal, intramuscular, intraperitoneal, intrapulmonary, intraspinal, intrasternal, intrathecal, intrauterine, intraurethral, intravenous, subarachnoid, subcapsular, subcutaneous, transmucosal, or transtracheal.

Targeted Contrast Compositions. In one embodiment, a targeting contrast agent comprising an imaging contrast agent composition and a quantity of death tags as described above may be used for detection and quantification of one or more death markers in vivo. Such detection and quantification can be used to diagnose the extent of cell death in a neoplasm or injured tissue.

One problem with designing new contrast agents for molecular imaging has been the lack of methods that provide information concerning contrast agents and their cell surface distribution and subcellular trafficking at the supramolecular level. The introduction of Electron Energy Loss Spectroscopic Imaging (EELSI) and Energy Dispersive X-Ray Spectroscopic Imaging (EDXSI) provided sensitive methods of molecular detection in situ. (Malecki 1996, Malecki et al 2002). In EELSI and EDXSI, genetically engineered antibodies tagged with atoms of selected exogenous elements can be localized within three-dimensional architecture of cells and cell organelles with atomic accuracy. In combination with rapid cryo-immobilization, information obtained from these imaging methods is similar to a life-like depiction of the events, wherein biochemical methods are limited by the possibility of translocation of probes or transmetallation of the reporter during the procedures. Therefore, the methods developed herein are advantageous because they reveal the molecular mechanisms governing bio-distribution and bio-compatibility. The targeted contrast described herein provides a similarly sensitive method for detecting such information in vivo.

According to some embodiments, a targeted contrast composition is provided comprising the death tags and reporters as described herein. The targeted contrast composition may be used with diagnostic imaging techniques such as X-ray, computed tomography (CT) Raman, MRI, NMR, USG, fluorescence, gamma-camera, SPECT, PET, and the like to provide a more accurate localization and diagnosis of dead cells (e.g., in treated malignant tumors or cells dying due to injury, pathological condition or ischemia) in a subject's body in vivo.

A general contrast agent or contrast media is a substance that is used to enhance the contrast of anatomical structures, cavities, or fluids within the body in diagnostic imaging techniques of radiology, sonography, nuclear medicine. Contrast agents are commonly used to enhance the visibility of blood vessels, urinary and the gastrointestinal tract by filling these structures with a substance creating better visibility than these structures alone. For additional guidance, see Textbook of Contrast Media 1^{st} Edition, (P. Dawson, D. O. Cosgrove and R. G. Grainger, eds.) Isis Medical Media Ltd., Oxford, UK, 1999, which is hereby incorporated by reference in its entirety as if fully set forth herein.

A targeted contrast is a substance that is guided to the specific molecules with the purpose to highlight only said molecules. In some embodiments described herein, a targeted contrast composition may be used to enhance visibility of dead cells that present markers of death. In one embodiment, the death-markers are phosphatidyl serines on surfaces of dying cells. In another embodiment, the death-markers are the dying cells' genomic DNA accessible due to broken membranes. In another embodiment, the death markers are the dying cells' histones, lamins, and/or intermediate filaments accessible due to broken membranes.

Examples of general contrast agents include, but are not limited to, barium, water, water soluble iodine, iodine mixed with water or oil, sterile saline, air occurring naturally or introduced into the body, paramagnetic substances. The type of contrast agent used can be classified, generally, based on the type of imaging technique used. Such techniques may include, but are not limited to X-ray based, magnetic resonance based or ultrasound based or injection of radionuclides. However, the injection of radionuclides does not provide any anatomical information, but rather, only provides a signal to show the distribution of the radionuclides, therefore requiring supplemental imaging techniques providing anatomical information.

Targeted Contrast Compositions for X-Ray-based diagnostic imaging. Iodine (I) and barium (Ba) are the most common types of contrast agents for enhancing X-ray based imaging methods such as radiography and CT. Various iodinated contrast media exist, with variations occurring between the osmolarity, viscosity and absolute iodine content of different agents. For example, contrast agents for X-ray based diagnostic imaging are based on tri-iodobenzene with substituents added for water solubility. Diatrizoate, an ionic corm, was introduced in 1954, but the high osmolality of this compound (1.57 osm kg-1 for a 300 mg l mr1 solution) was found to be the source of chemotoxicity. In the 1970s, a non-ionic form, iohexyl, lowered osmolality (0.67 osm kg1), and is still widely used today under the names Omnipaque® and Exypaque®.

Because osmolality was still excessive, a dimeric form was introduced, iodixanol (Acupaque® and Visipaque®; O. osm kg-1). Intravascular agents based on other mid-Z to high-Z elements have not been successful due to toxicity, performance or cost. The low molecular weights of the iodine agents (diatrizoate, 613; iohexyl, 821; iodixanol, 1550) effect rapid renal clearance and vascular permeation, necessitating short imaging times. Several other experimental X-ray based contrast materials are used as blood pool agents, including standard iodine agents encapsulated in liposomes, a dysprosium-DTPA-dextran polymer, polymeric iodine-containing PEG-based micelles, perfluoroctyl bromide, derivatized polylysine linked to iodine, and iodine linked to a polycarboxylate core (P743, MW=12.9 kDa).

Therefore, intra-arterial catheterization is commonly required, but carries risks of arterial puncture, dislodgement of plaque, stroke, myocardial infarction, anaphylactic shock and renal failure.

A major problem for developing contrast agents for X-ray or CT is that, even when conjugated with antibodies or other targeting moieties, they fail to deliver iodine to desired sites at concentrations which are high enough to make them detectable in molecular imaging. The present invention overcomes this major challenge.

In one embodiment, the metal nanoparticle tag associated with the nanoparticles used herein is gold. With a higher atomic number (Au, 79 vs. 1,53), and a higher absorption coefficient (at 100 keV: gold=5.16 cm2 g-1; iodine=1.94 cm2 g-1; soft tissue=0.169 cm2 g-\ and bone=0.186 cm2 g-1), gold provides about 2.7 times greater contrast per unit weight than iodine. Imaging gold at 80-120 keV reduces interference from bone absorption and takes advantage of lower soft tissue absorption which reduces patient radiation dose. Further, the higher molecular weight of noble metal nanoparticles permits much longer blood retention, so that useful imaging may be obtained after intravenous injection, likely obviating the need for invasive arterial catheterization for diagnostic triage. Other noble metals have similar advantages over iodine. According to some embodiments, molecular imaging with gold is possible because each nanoparticle bound to a targeting agent such as a death tag described above would deliver approximately 100-30,000 gold atoms to a death marker, which is multiplied by the number of markers of death on site, significantly increasing the signal without increasing the noise, thereby significantly increasing signal to noise ratio.

Targeted contrast compositions for magnetic resonance based diagnostic imaging. MRI is based upon non-ionizing radiation. Commonly used compounds for contrast enhancement for magnetic resonance imaging are gadolinium (Gd) based. Other superparamagnetic metals such as Eu, Fe, Ni and Co are also suitable for use with in vivo or in vitro MRI or in other in vitro methods such as nuclear magnetic resonance (NMR). Magnetic resonance based contrast agents alter the relaxation times of tissues and body fluids to which they are delivered. In particular, the agents affect T1 or T2 relaxation time of protons located nearby. Such registered contrast differences between various tissue compartments that are generated by local differences in relaxivities of water protons between those compartments translate into varying degrees of brightness of the image details on the MRI scanner's screen. Therefore, it is not the strength of the resonance signal itself, but rather the relative differences in signal intensity between various structures and/or in the signal to noise ratios that result in successful visualization of the analyzed features.

Iron nanoparticles have also been used successfully as magnetic resonance imaging (MRI) contrast agents.

Nevertheless, none of these contrast agents were designed, intended, considered, as targeted contrast specific to any marker of death.

Superparamagnetic metal atoms affect water proton relaxivity in their very immediate vicinity. 10⁻⁵ M or 100 microM of Gd is considered to be the threshold for inducing such a change in relaxivity of water, so that it will be detected in NMRor MRI. If chelated into a death marker binding domain as described herein (e.g., an scFv antibody fragment, sdFv antibody fragment, CDR antibody fragment, SDR antibody fragment, affibody, aptamer, or a complementary molecule targeting any death marker), these atoms will locally affect relaxivity, thus indirectly report the presence of death tags, thus indirectly they report localization and amount of death-markers i.e., the location of death. All together, they will also report the extent of cell death.

Previous attempts have been made to target selected living tissues (but not damaged or dead tissues) by randomly attaching reporters such as Gd chelates, dendrimers, or Fe nanoparticles to monoclonal IgG antibodies (e.g., Curtet et al. 1985, Mendonca et al. 1986, Linger et al. 1986, Weissleder 1991, Unger et al. 1999, Kobayashi et al. 2003). However, several factors have contributed to the failure of these attempts. First, random incorporation of reporters into IgG molecules leads to compromised specificity of antibodies with their denaturation, resulting in low specific binding signal and high background due to non-specific binding. This is also known as a “false positive” result. Second, the significant size of the IgG antibodies including the reporters as well as the changes in their properties due to the reporter incorporation results in steric hindrance and repulsion forces. Thus the targeted contrast was not able to reach and label the targeted structures. This is also known as a “false negative” result. Third, repetitive injections led to immunological responses, which interfere with the efficacy of the marker. Fourth, Gd that was released from the IgGs became involved in transmetallation processes, leading to serious toxicity to recipients. Fifth, release of Fe from iron-based superparamagnetic nanoparticles led to iron toxicity through oxidative stress. Sixth, the large size of IgGs limited the number of atoms that accumulated upon the target, resulting in a very weak signal.

A different approach to improving labeling effectiveness was generated by genetically engineering heterospecific, polyfunctional molecules as described and used herein. As described above, the death tags described herein are engineered to contain multiple highly specific, yet separate domains that are assigned to their functions. Such domains, as described above, may include a death marker binding domain (e.g., scFv fragments, sdFv fragments, CDR fragments, SDR fragments, CDs, Fab fragments, IgGs, IgMs, and IgAs), a reporter binding domain (e.g., a metal binding domain or MBD), and a reporter component (e.g., a metal nanoparticle). Upon incorporation of a superparamagnetic metal or upon linking a metal nanoparticle tag as a reporter, these death tags gain superparamagnetic properties without adversely affecting their targeting functions.

Targeted contrast compositions for ultrasonography (USG). By replacing the reporter molecules with microbubbles, the death tags may be detected using ultrasonography. A microbubble may be made of a hollow core and a metal shell, creating a core-shell “bubble” that responds strongly to the ultrasound waves.

Targeted contrast compositions for Positron Emission Tomography (PET). Presence of metal binding domains within the framework of the death tags described herein not only provides a binding site for superparamagnetic and noble metals as reporters, but also provides a binding site for radioactive isotopes emitting beta radiation (e.g., Cu64). This makes the death tags particularly useful tools for detection of death using PET. Further, presence of functional groups at the termini of the targeting domains in the death tag provides a binding site for the incorporation of ¹³¹I and ¹⁸F, which are alternatives for beta emitters.

Targeted contrast compositions for Gammascintigraphy and Single Photon Emission Tomography (SPECT). The presence of functional groups as described above may also be used to provide a binding site for gamma-emitting radiation (e.g. ¹²⁵I, ¹²³I, Tc99m, ¹⁵⁹Gd, and other radionuclides). This makes the death tags useful tools for the detection of death using gamma camera, gammascintigraphy and SPECT.

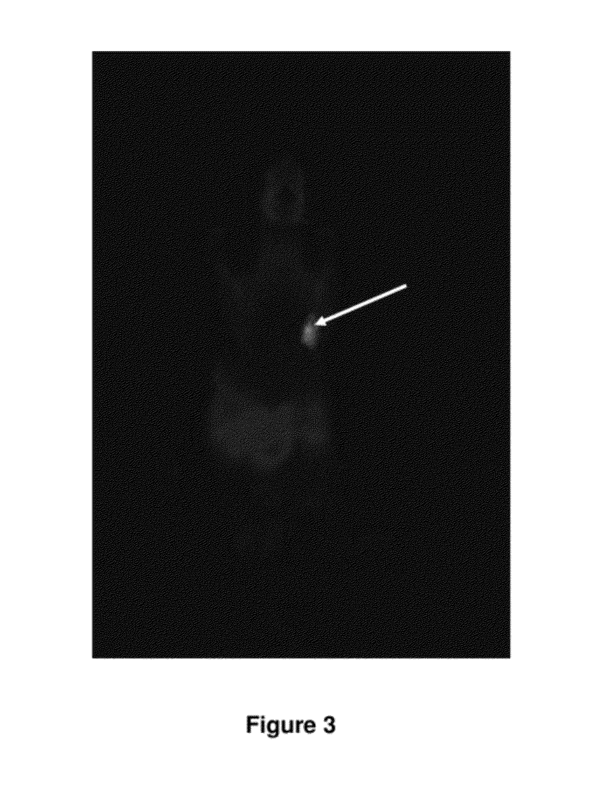

Targeted contrast compositions for fluorescence, infrared, Raman imaging. As shown in FIG. 3, death tags may also be designed to be suitable for imaging sections or specimens with various types of light, but may be missing the body heat spectrum. For imaging thicker structures and body heat, the long wavelength is suitable so it fits into an energy window suitable for detection of death tags.

In Vitro and Ex Vivo Use of Death Tags

Cancer cells that die or are dying spontaneously or from exposure to toxic therapy shed their cellular components into physiological fluids (e.g., blood, plasma, serum, lymph, cerebrospinal fluid or urine). These components include chromosomes and fragments thereof, genomic DNA, ssDNA, and dsDNA released into the circulation, present in a form of circulating free DNA (cfDNA). Detection of cfDNA is possible with the aid of death tags described herein. These death tags are an additional tool for discovering the existence, onset and advances of cancer that are difficult to detect or often go undetected by standard detection or diagnostic methods (e.g., ovarian or pancreatic cancers). One of the earliest markers in the formation of primary tumors and metastases is presence of cfDNA in blood or lymph of the patients. Further, detection of cfDNA in a physiological fluid can help in diagnosing cancer type based on organ-specific gene detection (e.g. cytokeratins). The detection of cfDNA is not an easy task, as evidenced by the low concentration (in the range of picograms) of cfDNA present in the physiological fluid. Anti-ssDNA and anti-dsDNA scFv, sdFv, CDRs, SDRs, complementary domains, ligands and other functional antibody fragments allow for capture and isolation of the cfDNA in the form of ssDNA, dsDNA and RNA from decaying cancer cells. This can be followed by DNA sequencing or RT from RNA followed by cDNA sequencing.

Therefore, in some embodiments, the death tags described herein may be used to detect cfDNA or cfRNA from decaying cancer cells in a physiological fluid sample of a subject who is suspected or diagnosed of having cancer, thereby diagnosing a cancer or a neoplasmic process. Physiological fluids that may be used in accordance with the embodiments described herein may include, but are not limited to, blood, plasma, serum, lymph, pleural fluid, peritoneal fluid, cerebrospinal fluid and urine.

In one embodiment, detection of cfDNA in a physiological fluid sample may be accomplished using one or more of the following steps. First, a death specific marker is chosen or identified. In one embodiment, the death-markers are ssDNA and dsDNA, histones, or lamins. Second, a death tag is selected to target the death specific marker. The death tag may include a death marker binding domain, a reporter binding domain and a reporter component. The death marker binding domain may be an antibody or functional fragment thereof, as described above. In one embodiment, the death marker binding domain is an scFv or sdFv or SDR or CDR. The reporter component provides a signaling presence and visualization of the location of the tag bound to the death marker in the dead cancer cell being genomic DNA (gDNA) or RNA. In one embodiment, the reporter is a metal nanoparticle tag, a fluorescent tag or ultrasound tag. Modification of a death marker by linking it with a reporter tag is further described above. The third step involves exposing a physiological fluid sample to the death tag and then isolating of the cfDNA, lamins, or/and histones bound by the tag for further analysis. These steps result in the detection of cfDNA in the samples drawn from a patient. The isolated cfDNA may be used for testing for cancer, or oncogene specificity.

In some embodiments, the studies described herein enable the use of superparamagnetic or noble metal of fluorochrome or microbubble linked death tags that target gDNA for the detection and diagnosing of cancer. The death tags also allow us to determine differences in intensity of cancer decay ex vivo.

In some embodiments, these death tags allow us to eliminate cfDNA from decaying cancer cells from circulation, thus reducing the risks of cancer induction in healthy cells. It has been reported that the presence of dsDNA sequences, with or without nucleoproteins, carries a risk of being incorporated into healthy cells. Incorporated dsDNA sequences may contain oncogenes, and although they are present in small amounts and internalized with low efficiency, these oncogenes may induce cancer. Therefore, in some embodiments, a method for elimination or removal of cfDNA such as ssDNA or dsDNA from the patient's circulation is provided to stop infiltration and metastasis of cancer after the death of cancer cells.

In some embodiments, the method for diagnosis, elimination or removal of cfDNA may be accomplished using an extracorporeal procedure. An extracorporeal procedure is a procedure in which blood is taken from a patient's circulation to have a process applied to it, ex vivo, before it is returned to the circulation. The apparatus carrying the blood outside of the body is known as the extracorporeal circuit, and diversion of a subject's blood flow through such a circuit that is continuous with the normal in vivo body circulation is known as an extracorporeal circulation. This procedure uses an approach that is similar to that used for dialysis in therapy of kidney diseases.

In one embodiment, a vascular access is established in a subject to establish the extracorporeal circulation. A vascular access is a site on the subject's body from which blood is removed and returned, and may include, but is not limited to, an arteriovenous (AV) fistula, an AV graft, or a venous catheter. Once a vascular access is established, it may be connected to a heparinized tube to establish the extracorporeal circulation.

In some embodiments, a subject having cfDNA or suspected of having cfDNA in their blood, is given an effective dose of death tags such that the death tags bind the cfDNA in the blood and are present in an extracorporeal circuit. The extracorporeal circulation may be exposed to a magnetic source such that the cfDNA bound to death tags are retained by the magnetic source, but the remaining blood returns to the general circulation.

The extracorporeal procedure may be carried out using a set of instruments that include a magnetic source (e.g., MRI, NMR or electromagnetic radiation), a pump to keep the extracorporeal circulation flowing (e.g. peristaltic pump) and an extracorporeal circuit (e.g., heparinized tubes).

Use of death tags for detection of non-cancerous cell decay and death (e.g., cardiac cell decay). In some embodiments, death tags may be used to detect non-cancerous cell decay and death. Non-cancerous cell decay and death may be detected by 1) the presence of cell-specific components of cell decay or death; or 2) a change in the level of cell-specific components of cell decay or death in a sample of physiological fluids (CF) ex vivo. In addition to cancer cell death, the methods described herein may be used to detect cardiac cell death due to myocardial infarction or brain cell death due to stroke or other ischemic events or traumatic injuries in particular internal body injuries or crashed limbs and other body parts. Dying cells release their cellular components that are often cell-specific, into the physiological fluids (CF). For example, cardiac myocytes release molecules of the contractile system (myosin, actin, tropomyosin, troponin), and fragments of the cardiac cytoskeleton (titin, nebulin, desmin) when they die. Detection of these molecules is possible with the aid of death tags. Therefore, these tags are an additional tool for determining whether a patient has had an occulted heart attack (or “silent heart attack”). These cytoskeletal and cytocontractile elements may be targeted in the emergency room as well by the in vivo methods described above when the injury is occurring or has recently occurred.

In vitro detection of death tags. In addition to the reporters described above, in vitro or ex vivo use of death tags used with a blood or physiological fluid samples may include fluorochromes that can be detected with spectrophotometer or ELISA reader, or a superparamagnetic reporter that can be measured with NMR or other reporter detecting modalities or with ultrasound to detect microbubbles, all guided by targeting domains of death tags.

Currently, commercially available probes have toxic effects, especially when used in long-term studies (Deo et al 2007, McKoy et al 2008). The embodiments described herein are non-toxic and have several advantages over the currently available probes. For example, the death tags described herein can:
be designed to label cells that are dead, dying, or in the final stages of death in addition to cells that are in the reversible stages of apoptosis, necrosis or any other type of cell disintegration;
generate a non-fading, stable signal;
bind with specificity to dead cells with little to no non-specific binding;
enhance the detection signal by labeling multiple sites or domains of target death markers because of their small size.

Quantitative Analysis of Death Tags.

In some embodiments, an injury or treatment may be analyzed and/or monitored by calculating a death ratio. A progressive injury or a response to toxic systemic therapy will manifest as increasing proportion of dead cells as compared to the healthy cells, i.e., a death ratio. When a tissue is healing, the volume of dead cells steadily declines and regenerating cells take the place of the dead ones. Therefore, ratio between intact or living and healing cells' volume and dead cells' volume will change depending on the dynamics of the disease or trauma or healing processes. This death ratio can also change based on progression of the disease or effects of therapy, and allows specific quantification of the pathology dynamics. For clinical purposes, this can be amplified by step-wise approaches involving usage of individual or pools of clones tags for death marker one after another. Dramatic increase of the signal recorded with CT or other molecular imaging/detecting modalities occurs.

Every cell contains approximately 6.6 pg of DNA. The smallest tumor clinically detectable contains approximately 10B cells, thus 6.6×10⁹ pg of DNA or 6.6 micrograms of DNA. This corresponds to 6.6 mM concentration of nucleotides. With each triplet targeted by a tag modified with 1000-30000 atoms of the reporter component (e.g., noble metal or superparamagnetic metal), the concentration of the reporter component far exceeds 2M. This is well within the reach of CT (noble metal) or MRI (superparamagnetic metal) or USG (microbubbles). Therefore, it is possible to image dead cells within a clinically detected tumor. If a particular therapy is successful and leads to the death of all cancer cells in this tumor, then the entire tumor will light up in CT after injecting an effective amount of death tags into the patient's body our tags of death or death tags. This approach also reveals the anatomical topography of the death within architecture of the neoplasm (completeness of cancer cells extermination) and within topographical anatomy of the patient.

The quantitative and qualitative differences discussed above can be determined with the aid of IgG, IgM, scFv, sdFv, Fab, CDs, CDR, SDR, and ligands directed against the molecules present on and/or in dead cells. Such determinations are important for making a clinical diagnosis with prognostic and therapeutic consequences. Prior to the current disclosure, these differences have been assessed in vitro using diagnostic histopathology and immunohistochemistry on frozen or paraffin sections. The current disclosure describes death tags to qualitatively and quantitatively determine these differences using diagnostic immunohistochemistry in vivo via assessment by CT, MRI, USG, PET, SPECT, RSI, FL, and the like.

A limitation in using targeted contrast agents in CT, MRI, fluorescence, or USG is a threshold of the contrast detection, which determines the sensitivity of detection. Several components contribute to the final sensitivity of detection, including the sensitivity of the instruments and sensitivity of the probes' reporters. Further, the specificity of the death tags determines the signal to noise ratio, which also contributes to the detection. The more reporters that bind to the target, the stronger the signal detected by the instruments. The more specifically they bind, the higher or better the signal to noise ratio. Small and specific scFv, CDR, SDR, or CD (e.g., 5-25 kDa) guided death tags can bind and accumulate on the target (e.g., 185 kDA receptor) in much larger numbers than those guided by large IgG (e.g., 155 kDa). Any increase in the number of targeted molecules or number of reporter component atoms per death tag would push the detection threshold into millimolar range no more than one IgG would label one death marker because of the steric hindrance; Malecki et al. 2002). For broadcasting prognosis and planning therapy, it is important to determine death marker density on or in the dying or dead cells.

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinary skill in the art and are described in numerous publications. Further, all references cited above and in the examples below are hereby incorporated by reference in their entirety, as if fully set forth herein.

### Example 1

### Generation of scFv, sdFv, CDR, SDR, and CD as Death Marker Binding Domains of Death Tags Targeting Death Markers on/in Dead and/or Dying Cells

To generate a death tag for use in molecular imaging of cell death in vivo, a death marker binding domain with high specificity and affinity of the domains targeting death tags to the molecular markers of the death is made. Designing, engineering, and manufacturing such death marker binding domains may be accomplished as follows.

scFv, sdFvs, CDRs, SDRs, and/or complementary domains (CD) against ssDNA and dsDNA, histones, and lamins were constructed from the DNA libraries as described below followed by selections through generating combinatorial mRNA displays according to published protocols (Roberts and Szostak 1997, Wilson et al 2001, Shoemann and Traub 1990, Malecki et al 2002).

All the procedures started with B cells, which were isolated from blood drawn from Systemic lupus erythematosus (SLE), Chronic Arthritis, Antiphospholipid Antibody Syndrome (AAS), and cancer patients. The patients' blood was drawn as small aliquots under the informed consent based upon the IRB approved protocol. For each sample, 2 ml of balanced salt solution was added and mixed with 2 ml of anticoagulant-treated blood to dilute the blood. Each of the diluted blood samples (4 ml) were layered on top of 3 ml of Ficoll-Paque Plus in a Falcon tube without mixing. The samples were centrifuged at 400 g for 30-40 minutes at 18-20° C. This led to separation of the sample into four layers: 1. plasma (top), 2. lymphocytes, 3. Ficoll-Paque Plus, and 4. granulocytes, erythrocytes. After discarding the plasma, the lymphocyte layer was transferred to the new Falcon tube, to which at least 3 volumes of balanced salt solution were added and mixed. The sample was centrifuged at 400 g for 10 minutes at 18-20° C. The supernatant was removed. The lymphocytes were resuspended in 6-8 ml balanced salt solution. The cells were counted on the Beckman Coulter cell counter with forward scattering indicative of cells' sizes and side scattering indicating their viability. The viable cells were sorted and used for creating cDNA library for production of anti DNA, antiHistone, and antiLamin antibodies.

To ensure viability, the B cells were isolated by negative selection. Non-B cells, i.e., T cells, NK cells, monocytes, dendritic cells, granulocytes, platelets, and erythroid cells depletion was performed with antibodies against CD2, CD14, CD16, CD36, CD43, and CD23 tagged with our magnetic beads. This left the sample with a pure population of untouched B cells. This was validated by labeling of B cells with CD19 and CD20. The samples were further processed or stored in liquid nitrogen.

After extracting total RNA from the isolated lymphocytes Trizol (MRC) according to published protocols (Chomczynski et al 1991), RT-PCR was performed to amplify human antibody variable fragments. cDNA was prepared using SuperScript™ III First-Strand Synthesis System (Invitrogen). Alternatively, cDNA was obtained by Cells-To-cDNA kit from Qiagen. Approximately, 5 pg to 25 pg of RNA or mRNA was reverse transcribed into the first-strand cDNA using short, degenerate primers designed with help of framework sequences in public domain (Johnson and Wu 2004).

DNA sequences coding light chains (LC) and heavy chains (HC) were amplified using standard protocols and sequenced. The primers for this step were designed to have extensions for SfiI and SacI restrictions sites Sfi I: 5′ GGCCNNNN*NGGCC . . . 3′ (SEQ ID NO:51); SacII: 5′ CCGC*GG . . . 3′ (SEQ ID NO:52). The amplicons were run on 2% agarose gel, stained with SybrGold, and imaged with Storm 840. DNA sequences coding LC and HC were amplified using standard protocols. After digestion and clean up, the amplicons were assembled into the DNA constructs coding for single chain variable fragments (scFv), single domain variable fragments (sdFv), complementary domain regions (CDRs), or complementary domains (CDs).

mRNA displays. Selections of the clones were performed using mRNA display technique in the details published (Roberts and Szostak 1997, Wilson et al 2001). However, the significant modification was introduced at the final stage of the protocol dealing with targeting according to the protocol described in the details (Malecki et al. 2002). Briefly, the human sonicated DNA fragments were extended with incorporation of biotin or digoxigenin charged nucleotides. Upon completion of forming mRNA*scFv complexes, monovalent scFv targeting biotin or digoxigenin, which were tagged with superparamagnetic or noble metals were introduced. After 15 min at room temperature, the clusters were fished out using magnets (when using superparamagnetic nanoparticles scFv) or spinning (when using noble metal nanoparticles). The rest of the procedure involved RT PCR and cloning of the sequences showing high affinity towards DNA. For selection of antiLamin and antiHistone clones, the lamins and histones were isolated as described above and were modified by introducing biotin or digoxigenin tags for selection procedures performed like those for DNA described above.

### Example 2

### Generation of Death Tags Incorporating Noble Metals

To ensure the bio-safety, sensitivity, and accuracy of the death tags used in vivo as described herein, a stable link between death marker targeting domain and a reporter molecule such as a noble metal atom was accomplished by designing and engineering various metal binding domains (MBD), including binding domains of noble metals (e.g., Au) and paramagnetic and/or their salts (e.g., Gd, Eu, Fe, Tb, iron oxides, or other suitable metals as described above) and for nanoparticles assembled into the core-shell. Exemplar binding domains are listed below:

(SEQ ID NO: 33)
(Gly-)n-CyS
(SEQ ID NO: 34)
(Gly-Arg-)n-Cys
(SEQ ID NO: 35)
(Gly-Lys-)n-CyS
(SEQ ID NO: 36)
(Gly-Asp-Gly-Arg)n-Cys
(SEQ ID NO: 37)
(Gly-Glu-Gly-Arg)n-Cys
(SEQ ID NO: 38)
(Gly-Asp-Gly-Lys)n-Cys
(SEQ ID NO: 39)
(Gly-Glu-Gly-Lys)n-CyS

B Binding Domains Suitable for BNT:

MAP16-B

Gd or Eu Binding Domains Suitable for Gd MRI and NMR and Death Tag Guided Therapy:

(SEQ ID NO: 40)
(Glu-Glu-Glu-Glu-Glu)n
(SEQ ID NO: 41)
(Glu-Glu-Glu-Glu-Glu-Glu)n
(SEQ ID NO: 42)
(Asp-Asp-Asp-Asp-Asp)n
(SEQ ID NO: 43)
(Asp-Asp-Asp-Asp-Asp-Asp)n
(SEQ ID NO: 44)
Phe-His-Cys-Pro-Tyr-Asp-Leu-Cys-His-Ile-Leu

Ni and Co Binding Domains:

(SEQ ID NO: 45)
(Gly-Asp-Gly-Arg)n-(His)5,6
(SEQ ID NO: 46)
(Gly-Glu-Gly-Arg)n-(His)5,6
(SEQ ID NO: 47)
(Gly-Asp-Gly-Lys)n-(His)5,6
(SEQ ID NO: 48)
(Gly-Glu-Gly-Lys)n-(His)5,6
(SEQ ID NO: 49)
(Gly-Arg-)n-(His)5,6
(SEQ ID NO: 50)
(Gly-Lys-v-(His)5,6

Beckman BIOMEK FX Span-8 and 96 Channel Robotic System was loaded with each of the domains within a separate channel. In particular, one of the channels contained the noble metal nanoparticles (e.g., gold) or superparamagnetic core shell nanoparticles or microbubbles or fluorochromes. Each of these domains contained a functional domain at the amino or carboxyl terminus as detailed below. The sequence of the processing allowed addition of the single domain to a single particle at a time. Alternatively, a microfluidic system was used with the identical aim. As a result, heterospecific mono-, di-, tri-, poly-mer scFv, sdFv, CDR, SDR, CD guided death tags were easily assembled and tested, while firmly anchored to the nanoparticles as the core structure. Some constructs led to expression of fusion proteins, but their MBD at the carboxyl or amino terminus served as the anchors to the nanoparticles.

Manufacturing of pure noble metal nanoparticles. Nanoparticles derived from noble metals Au, Pt, Pd and Ag were generated by laser ablation of 99.99% purity metal foils in a chamber filled with deionized water under continuous flow as described previously (Malecki 1996). Some variability in sizes was compensated by gradient ultracentrifugation, which also resulted in their condensation.

Death tags charged with noble metals and guided by targeting domains. Plasmid constructs were generated as described previously (Malecki et al. 2002). Briefly, death marker binding domain constructs having coding sequences that generate antiDNA (anti-ssDNA or anti-dsDNA), antiHistone, antiLamin or antiPS molecules such as CDR, SDR, scFv, sdFv, or CDs as described above were generated. The constructs may be one or more of SEQ ID NOs: 1-32.

Chelating sites fused with death marker binding domains were then covalently bound to gold nanoparticles to form gold-linked death tags. While the current examples provide for the production of gold nanoparticles, nanoparticles using other noble metals (e.g., Pt, Pd, Ag) may be successfully manufactured according to previously developed methods well known to the technicians skilled in the art (Malecki 1996). Purification of the gold-charged death tags was accomplished using affinity and size exclusion chromatography columns.

Determination of noble metal atoms per nanoparticle and number of nanoparticles per tag. The number of atoms per nanoparticle was determined by measuring the diameter with FEEFTEM (Titan) or EFTEM (LE0912) or FESTEM (HB501) at zero loss followed by measuring MDN with EDX and/or EELS of the beam parked over the nanoparticle using the Si drifted detector or ccd chip (Noran, Zeiss or Gatan, respectively). The ratios of nanoparticles to scFv, sdFv, CDR, CD, IgM, IgG, Fab was determined by ratios between the noble metal nanoparticle and carbon counts from EDX and EELS in Zeiss 912 or Titan or VG equipped with Zeiss or Gatan software.

### Example 3

### Generation of Death Tags Incorporating Superparamagnetic Reporters

To ensure the bio-safety, sensitivity, and accuracy of the death tags in vivo using nuclear magnetic resonance techniques as described herein, a stable link between death marker targeting domain and a reporter molecule such as a superparamagnetic atom was accomplished by designing and engineering various specific metal binding domains (MBD).

Plasmid constructs were generated as described above previously described (Malecki et al. 2002, Szostak et al. 2005). Coding sequences for ssDNA and dsDNA were selected from the surface displayed libraries cloned into pM vectors designed with CMV immediate early promoter, SV40 poly(A) termination, and neomycin-resistance. The constructs were expressed in cell free systems or electroporated or lipofected into human myelomas, CHO and/or HEK293. Expression of these constructs resulted in the secretion of ready fusion proteins. In some cases, these proteins were exposed to a couple of rounds of de- and re-naturation processes by exposing them to high pressure freezing at 3000 mbar, -196 deg C. Chelating sites were saturated with metal ions: Gd, Eu, Fe, Ni and Co. Alternatively, the iron oxide nanoparticles were coated with shells of noble metals. They were linked to fusion proteins involving protocols identical to those as used for noble metals. Purification from non-bound metal was performed on affinity columns. The myelomas were cultured in modified roller bottles (Sigma) or bioreactors (New Brunswick) according to standard protocols. Alternatively, cell free expression systems were used according to standard protocols.

Determination of metal atoms incorporated into chelating sites. The chelating sites of MBD were saturated with Gd or other superparamagnetic ions. Subsequently, these samples were purified on the affinity columns. Finally, they were analyzed with electron energy loss spectral imaging (EELS) and x-ray dispersive spectroscopy to determine total C to Gd metal atom ratio or, in other words, the number of incorporated atoms per death tag molecule.

Alternatively, the death tags were altered through amine or carboxyl terminus modification with I. Subsequently, these samples were purified on the gels. They were analyzed using ratios between I and C using EDX and EELS.

Alternatively, the death tags were altered through amine or carboxyl terminus modification involving insertion of MBD and linked with noble gold metal or core/shell clusters. Subsequently, these samples were purified on the size exclusion chromatography columns. They were analyzed using ratios between I and C using EDX and EELS.

### Example 4

### Validation of Death Tags in Detecting Cancer Cells In Vivo or In Vitro

The following materials and methods are used for the validation experiments described herein, but also apply to the experiments described in all other Examples.

Cell cultures. Many cell lines have been used to test the death tags described herein. Exemplar cell lines were grown in media recommended by ATCC in incubators (New Brunswick, Fisher, Napco) in saturated humidity, 37 deg C., 5% CO₂, 0.5-5% O₂ (Table 2). As shown in Table 2 below, the cell lines were selected to cover the entire spectrum of primary cancer lineages (including cancers of ovary, testis, brain, lung, and pancreas). On all these cell lines, dead and dying cells were efficiently detected and highlighted with the death tags described herein. All cell lines were obtained from ATCC unless otherwise noted.

TABLE 2
Cell Lines

| | | ATCC | |
|---|---|---|---|
| | Designation | Number | Other |
| Ovarian | TOV-112D | CRL-11731 | HER2 positive |
| cancer cell | OV-90 | CRL-11732 | HER2 positive |
| lines | OVCAR-3 | HTB-161 | ER positive, |
| | | | PR positive, |
| | | | AR positive |
| Breast cancer | SKBR3 | HTB30 | HER2 positive |
| cell lines | UACC-893 | CRL-1902 | HER2 positive |
| | UACC-812 | CRL-1897 | HER2 positive |
| | HCC1954 | CRL-2338 | HER2 positive |
| | AU565 | CRL-2351 | HER2 positive |
| | MDA-MB-453 | | HER2 positive |
| | BT474 | HTB-131 | HER2 positive |
| | HCC2157 | HTB20 | HER2 positive |
| | HCC2218 | CRL2340 | HER2 positive |
| | BT483 | CRL2343 | HER2 positive |
| | MCF7 | HTB-121 | HER2 positive |
| | | HTB22 | HER2 positive |
| | HCC1008 | CRL 2320 | HER2 positive |
| | HCC38 | CRL2314 | HER2 positive |
| | HCC70 | CRL2315 | HER2 positive |
| | HCC1143 | CRL2321 | HER2 positive |
| | HCC1187 | CRL2322 | HER2 negative |
| | HCC1395 | CRL2324 | HER2 negative |
| | HCC1419 | CRL2326 | HER2 negative |
| | HCC1428 | CRL2327 | HER2 negative |
| | HCC1500 | CRL2329 | HER2 negative |
| | HCC1569 | CRL2330 | HER2 negative |
| | HCC1599 | CRL2331 | HER2 negative |
| | HCC1937 | CRL2336 | HER2 negative |
| | HCC2218 | CRL2343 | HER2 negative |
| | Nm2C5 | CRL2918 | HER2 negative |
| | Nm2C5 gfp | CRL2919 | HER2 negative |
| | M4A4 | CRL-2914 | HER2 negative |
| | MDA-MB-330 | HTB127 | EGFR positive |
| | MDA-MB-468 | HTB132 | EGFR positive |
| | MDA_MB_231 | HTB26 | |
| | | | EGFR positive |
| | | | EGFR positive |
| | | | EGFR positive |
| Brain cancer | DBTRG-05MG | CRL-2020 | glioblastoma |
| cell lines | U-87 MG | HTB-14 | glioblastoma |
| Testicular | Tera-1 | HTB-105 | testicular |
| cancer cell | | | cancer |
| lines | | | |
| Lung cancer | NCI-H23 | CRL-5800 | HER2 positive; |
| cell lines | | | EGFR positive; |
| | NCI-H146 | HTB-173 | |
| | NCI-H345 | HTB-180 | EGRF and TGF |
| | | | positive; |
| | | | bronchial ducts |
| | HBE135 E6E7 | CRL2741 | EGFR positive |
| | HCC827 | CRL2868 | EGFR positive; |
| | | | Mutation A750del |
| | | | in EGFR |
| | HCC2935 | CRL2869 | EGFR positive; |
| | | | Mutation A751del |
| | | | in EGFR |
| | HCC4006 | CRL2871 | EGFR positive; |
| | | | Mutation A751del |
| | | | in EGFR |
| Pancreatic | MIA PaCa-2 | CRL-1420 | |
| cancer cell | PANC-1 | CRL-1469 | |
| lines | | | |
| Prostate | PC3 | CRL1435 | |
| cancer | 2G3 | CRL2435 | |

Several of the cell lines used in the experiments described herein are further described below in more detail. The cell lines TOV-112D CRL-11731 and CRL-117320V-90 were derived from primary malignant adenocarcinomas of the ovary at grade 3, stage IIIC. They were cultured in a 1:1 mixture of MCDB 105 medium and Medium 199, 85%; donor bovine serum 15% (ATCC). The cells were tumorigenic in nude mice. They formed colonies and spheroids when cultured in soft agar. The cells tested positive for HER2/neu and p53 mutation.

The cell line NIH OVCAR-3 HTB-161 was derived from the cells in ascites of a patient with malignant adenocarcinoma of the ovary. The cell line was grown in RPMI-1640 Medium (ATCC) supplemented with 0.01 mg/ml bovine insulin and donor bovine serum to a final concentration of 20%. The epithelial cells were positive for estrogen and progesterone receptor. They formed tumors in nude mice.

The cell line CRL-2340 HCC2157 was derived from the ductal carcinoma of the mammary gland tumor classified as TNM stage IiIA, grade 2, with lymph node metastasis. The cells were grown in a 1:1 mixture of Ham's F12 medium with 2.5 mM L-glutamine and Dulbecco's Modified Eagle's Medium adjusted to contain 1.2 gIL sodium bicarbonate with additional supplements (ATCC).

The cell line MCF7 HTB-22. The cells are positive for estrogen receptor and express WNT7B oncogene. The medium to culture this cell line is Eagle's Minimum Essential Medium (ATCC) with these added components: 0.01 mg/ml bovine insulin; donor bovine serum to a final concentration of 10%.

The cell line 184A1 CRL-8798 was originally established from normal mammary tissue and was transformed to benzopyrene. The line appears to be immortal, but is not malignant. The line grows in Mammary Epithelial Growth Medium (MEGM) (Clonetics) supplemented with 0.005 mg/ml transferrin and 1 ng/ml cholera toxin.

The normal, adherent fibroblast cell line Detroit 573 CCL-117 was derived from skin. It is grown in Minimum essential medium (Eagle) in Earle's BSS with nonessential amino acids (ATCC), sodium pyruvate (1 mM) and lactalbumin hydrolysate (0.1%), 90%; fetal bovine serum, 10%. The cells were grown into spheroids within a synthetic extracellular matrix.

Viability tests and doubling times. The cells were stained with Hoechst vs PI and counted on Beckton Dickinson or Beckman Coulter flow cytometer to determine ratios between total number of cells and dead cells at 24 hour intervals to determine doubling times and viability.

Selection of clones with high metastatic potential. For the in vitro studies described herein, cell lines described above were grown as described above. They were resuspended and spilled over the endothelial cells grown over extracellular basement membrane as described previously (Malecki et al. 1989). After short incubation at 37° C., the cells cultures were rinsed with media, while removing non-adherent cancer cells. The attached cells were resuspended again and split into single clones grown in multiwell plates. These enriched clones were used for further studies because they imitated the metastatic clones of the lines derived from the primary tumor.

Immunolabeling. Cell spheroids grown in the culture were spun down at 300×g. The cells were resuspended in the donor serum or whole blood to which superparamagnetic scFv were added. Upon completion of labeling, the cells were rinsed with PBS. They were studied with CT, MRI, USG, FL, RSI, PET, SPECT, or NMR or alternatively processed by freezing in preparation for laser scanning confocal microscopy (LSCM) or EDXSI or EELS. Alternatively, cell lysates were electrotransferred onto PVDF membranes and immunolabeled with guided death tags with or without chelated metal atoms.

Freezing and freeze-substitution of cell spheroids. The details of cryoimmobilization of cultures of cell spheroids by freezing are described previously and are only briefly presented here (Malecki 1992). Briefly, cells were injected into chambers were rapidly frozen in nitrogen slurry down to down to −196° C. The frozen samples were placed into methanol that was precooled to −90° C. in the freezer (ThermoNoran). Temperatures were maintained at −90° C., -35° C., and 0° C. for 48 hours. Infiltration with Lowicryl preceded polymerization with UV at −35° C. and ultramicrotomy. Alternatively, critical point drying was followed by fast atom beam sputter coating (IonTech).

Native electrophoresis. A 2% agarose gel was poured using a 10 mM Tris, 31 mM NaCl buffer of varying pH that did not contain any denaturing agents. The samples in their native state were loaded after being mixed with glycerol to add density without denaturing the proteins. The gel was run in the same buffer used for pouring the agarose at 60 mAmps until the desired separation was reached as determined by the presence of fluorescent markers with a molecular weight higher and lower than the scFv tested. The gel was then stained for 30 minutes in Sypro Tangerine Gel Stain (Invitrogen) diluted in the running buffer before imaging using a FluorImager (Molecular Dynamics).

SDS-PAGE. Electrophoresis was run on 12% polyacrylamide gel. Several 0.75 thick combs with the 2 mm lanes were loaded with standard, cell culture lysates. The samples, after mixing with SDS and DTT containing sample buffers (Sigma) were loaded into the wells. The gels were run using a Tris/Glycine/SDS/DTT running buffers. After the run, the gels were stained with colloidal silver or Sypro Tangerine for imaging using Storm 840 or FluorImager (Molecular Dynamics).

Electrotransfer. After electrophoresis, the samples were immediately transferred onto PVDF. The immunoblotting was performed with the Mini Trans-Blot Cell (Bio-Rad) within CAPS: 10 mM 3-[Cyclohexylamino]-1-propanesulfonic acid (CAPS), Tris/glycine transfer buffer 25 mM Tris base, 192 mM glycine, pH 8.3. Prior to the transfer, the cooling units were stored with deionized water at −20 C. Immediately after electrophoresis the gel, membrane, filter papers and fiber pads were soaked in transfer buffer for 5-10 minutes.

A tube or plate containing an aliquot of a patient's blood supplemented with superparamagnetic-death tags may be placed in the magnetic field of a magnetic field generator. Labeled cells, chromosomes and DNA were attracted and retained by the magnetic field generator while the blood was not. After rinsing with PBS, the labeled cancer cells were retained for further studies on the counting chamber, fluorometer, and/or confocal microscope.

Further, an extracorporeal method as described above may be used to separate death markers from blood of the patient. This method reduces the risk of healthy cells being transduced with oncogenes from cfDNA of decaying cancer cells.

Laser scanning confocal microscopy. (LSCM) The three-dimensional stacks of the cells labeled with death tags were imaged with the Olympus or Leica laser scanning confocal systems. Excitation wavelengths were used: 337, 488, 543, and 588 nm. Alternatively, reflected or Raman optics were used. Images were acquired with Kernel filtration and deconvolution of the data was followed by 3D or cascade display for analysis.

Spectral Mapping Using Energy Dispersive X-Ray Analysis Spectroscpic Imaging (EDXDI) and Electron Energy Loss Spectroscopic Imaging (EELSI). Supramolecular architecture analysis of the death tags was performed with Field Emission Scanning Electron Microscope with Energy Dispersive X-Ray Spectral Imaging System (EDXSI)-Hitachi 3400. Complete elemental spectra were acquired for every pixel of the scans to create the elemental databases. From the spectra, after selecting an element specific energy window, the map of this element atoms distribution was extracted and ZAF correction calculated (NIST). As death tags were tagged with superparamagnetic metal particles (nanoclusters or core-shell nanoparticles) or noble metal nanoparticles were tagged or incorporated into their structures, their location was determined based upon spectral elemental maps superimposed over molecular architecture with zero loss or carbon edge tuning (Malecki 1996, Malecki et al 2002).

Purity of elemental composition and geometry of gold nanoparticles were evaluated with EOXSI using Vacuum Generators 501, Hitachi S900, and JEOL 1540 instruments under control of Gatan, Voyager software.

X-ray, atomic absorption spectroscopic, surface plasmon resonance detection, centrifugation, and selection. One molecule of death tags (one gold nanoparticle ˜100-1000 atoms of gold, diameter ˜1.59 A; mass ˜197 amu each) increased the mass of scFv tagged by up to 19,966 and that was made of about 1000 atoms up to 196,667. Separation of death markers from blood via centrifugation was accomplished by centrifugation at low g, wherein the labeled cells fell to the bottom with respect to the unlabeled cells. This lead to rapid separation of death markers from the aliquot of the patient's blood.

CT—Computed x-ray Tomography. For evaluating relative contrast agents in CT, solutions of 1M, 0.1 M, 0.01 M, and 0.001 M, 0.0001 M sodium iodide, calcium chloride, gold chloride, and gold nanoparticles of various sizes in deionized water were dispensed into the wells of microarray plates. Additional rows contained blood, physiological saline, while an additional row was left empty, i.e., to contain air.

Computed tomography was pursued with Toshiba Aquilion 64-slice clinical scanner. Initial settings were as follows: voltage 120 peak kV, current 40 mA, exposure time of 0.6 s, slice setting 0.5 mm (the slices that were thereafter compressed into 2 mm display images), (modifications of these settings were indicated in the figure legends). ImageQuantTL® version 1.1.0.1 was used to evaluate relative peak pixel intensity of the samples on the computed tomography images utilizing a 0 to 255 level grayscale. The Aquilion scanner may also record phantoms for use in detecting biomarker density by measuring the signal intensity of the death tags in Haunsfield units (see, e.g., FIG. 18).

Nuclear magnetic resonance and selection. The wide-bore nuclear magnetic resonance (NMR) spectrometer operated at 9 T (Brucker) with a mouse-cage resonator was used to evaluate relative relaxivity of the samples based upon T1 measurements. T1 spin lattice relaxation time calculated using inversion recovery pulse sequence was measured using inversion recovery imaging with TI=50-4000 ms in 100 ms increments. T1 was also calculated from T1-weighted fluid-attenuated inversion recovery (T1-FLAIR) sequence (TrITe/Flip=2210/9.6/90), as well as standard T1 weighted imaging sequences (TrITe/Flip=400/6/90).

For studies of presence of products of cell decay in vitro, NMR spectrometers were used a small table top 0.5 T or 1.5 T (Bruker or GE) or 4.7 T, 9 T, and 11 T (Bruker). After labeling with superparamagnetic scFv, the blood sample containing labeled cancer cells was injected into microfluidic channel of 20 micron in diameter, which was placed with the field. Passage of the single cell, which was labeled with superparamagnetic scFv, was determined by the spectral response and recorded.

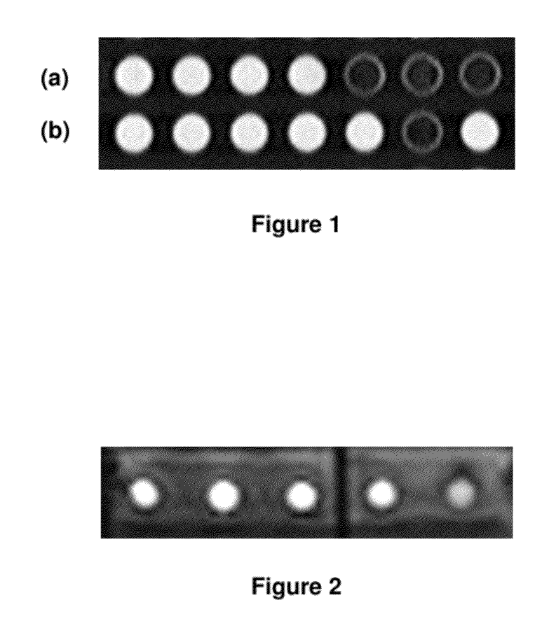

Several cancer cell lines were grown in extracellular matrix and exposed to radiation therapy (1-6 Gy) or chemotherapy (Cisplatin, Dexamethasone). To validate the effects of these therapeutic endeavors causing cancer cell deaths, we labeled treated cells with death tags charged with gold and imaged the samples within CT. Each well contained a different cell line. They were labeled with the antiDNA charged with gold clusters (Au*death tags). Immersed in serum, they were imaged with CT to determine the level of gene expression product for each cell line. Results are shown in FIG. 1-2.

Brighter spots are indicative of a higher number of death markers labeled with death tags charged with Au, which is equivalent to stronger effects of therapy. This is a much more accurate determination of cell deaths, than that used in clinical practice based upon annexin, because annexin only labels cells in the initial stages of apoptosis, which is a recoverable condition. Computed tomography was pursued with Toshiba Aquilion 64-slice clinical scanner. Initial settings were as follows: voltage 120 peak kV, current 40 mA, exposure time of 0.6s, slice setting 0.5 mm (the slices that were thereafter compressed into 2 mm display images). ImageQuantTL® version 1.1.0.1 was used to evaluate relative peak pixel intensity of the samples on the computed tomography images utilizing a 0 to 255 level grayscale.

In another experiment, ovarian cancer cells were grown and treated as above. Effectiveness of therapy was validated in MRI. For this purpose, the dead cells were labeled with superparamagnetic death tags.

Labeling of dead cells with death tags changed their properties, while making them susceptible to magnetic field. The more dead cells that were present, the more death biomarkers were accessible and able to be labeled in the form of exposed genomic DNA, the more death tags by anti-ssDNA and anti-dsDNA targeting domains. An increase in labeling of dead cells resulted in a higher relaxivity and brighter signal from the areas occupied by the dead cells labeled with death tags within MRI.

To summarize, significant differences were noticed in the signal strength generated between unlabeled ECM, fibroblasts, ovarian and breast cancer cells after labeling with superparamagnetic death tags. Moreover, the signal strength generated in 0.5 T NMR was sufficiently strong to detect passage of a single cancer cell through the microfluidic channel or micropipes.

### Example 5

### Isolation of DNA Using an scFv that Targets dsDNA

After cells die, elements of the dead cells (e.g., dead cells themselves, DNA, chromosomes, histones, and their deteriorated fragments) are released in the circulation, cerebrospinal, peritoneal or pleural fluids. As described below, single chain variable fragment (scFv) antibodies that target dsDNA were genetically engineered and used to pull out entire chromosomes after disrupting living cells. This scFv targeting dsDNA may also be used to detect and isolate the chromosomes, chromosome fragments and cfDNA (e.g., dsDNA) from the fluids or tissues obtained by fine needle aspiration (FNA). These scFvs were used to isolate and amplify DNA from cells and physiological fluids, validating their ability to detect the presence of genomic or cfDNA in such samples.

Anti-dsDNA single chain variable fragments (scFv_{dsDNA}). Single chain variable fragment antibodies (scFv) were genetically engineered as described below.

Briefly, fresh blood was received from cancer patients with Institutional Review Board (IRB) approval and with Informed Consent Forms (ICF) signed. White blood cells (WBC) were isolated using Ficoll-Hypaque technique. B cells were labeled with sorted chromosomes that were sorted with MACS (if the chromosomes were tagged with superparamagnetic) or FACS (if the chromosomes were tagged with fluorochrome). RT PCR was performed on each cell carrying dsDNA targeting variable fragments and the variable fragments were amplified and cloned within pM vectors as described below.

cDNA was generated using random hexamers (Intergrated DNA Technologies, Coralville, Iowa) and reverse transcriptase (Promega, Madison, Wis.) in reactions involving denaturing RNA at 70 deg C. followed by reverse transcription carried at 42 deg C. for 15 min. The cDNA quality was tested by the polymerase chain reaction (PCR) of beta actin and GAPDH as reference genes with the commercially available primers (ABI, Foster City, Calif.). For amplification of variable fragments, the primers sets were designed using the Kabat database. They were synthesized on 380 A DNA Synthesizer (ABI, Foster City, Calif.). The variable fragments were amplified with polymerase chain reaction using the mix of the generated cDNA, the synthesized primers, dNTPs, and Taq DNA polymerase (Hoffmann-La Roche, Basel, Switzerland) using the Robocycler (Stratagene, San Diego, Calif.).

The blunt ended amplicons were inserted into a pM construct containing the single dsDNA target sequence and a coding sequence coding for the dsDNA scFv. The DNA plasmid construct also contained metal binding domains capable of chelating superparamagnetic and fluorescent metals as described herein. The constructs were electroporated and expressed in human myelomas. The expressed clones were labeled in liquid phase with the transgenic receptors, which were modified with fluorescent reporters generating 545 nm and 619 nm emissions. The clones expressing the VH and VL chains were selected on the cell sorter FACS Calibur (Becton-Dickinson, Franklin Lakes, N.J.). The coding sequences of the selected clones were ligated by inserting the Gly-Ser linker coding sequence through overlap extension. The new constructs were also expressed in human myelomas. The coding sequences were verified after total RNA extraction, reverse transcription, amplification, and sequencing of amplicons on the ABI 3130XL or Junior DNA Sequencer (ABI, Foster City, Calif.).

Primary Cultures of Ovarian Cancers. After performing a surgical biopsy and/or paracentesis, followed by an evaluation by surgical pathologist on site, the cells were collected into the Dulbecco Modified Essential Medium within cell culture flasks. The growing ovarian cancer cultured cells (OCC) were maintained within the cell culture incubators at 37 deg. C., saturated humidity, and mixtures of CO2/02 gases (New Brunswick). The cells expressed 0.03-3 million EGFRwt or EGFRvIII per cell. The viability of the cells was determined by labeling with LIVE/DEAD® Viability/Cytotoxicity Kit for mammalian cells (Invitrogen, Carlsbad, Calif.) and flow cytometry and sorting on FACS Calibur or FACS Vantage SE (Becton-Dickinson, San Jose, USA).

The cells in suspensions from effusions or dispersed tumors were labeled with fluorescent or superparamagnetic antibodies and dispensed one EGFRvIII or EGFRwt positive cell per well by FACS or MACS. The cells were gently swollen by adding drops of 0.075 M KCl containing 0.1 μM colchicine and incubating for ½ to a couple of hours. The cells from clonogenic cultures were incubated with the media containing 0.1 μM colchicine for 1-16 h. That followed by spinning the cell clones within the agar at 100 g for 10 min at 37 deg C. and removal of the supernatant at the end of the spin. The cells were then swollen as described for the effusions. The cells were then exposed to the couple seconds long bursts of the low frequency ultrasonic waves (Bransonic, Danbury, Conn.), which were releasing the chromosomes. The quality of the chromosome architecture prepared this way was far superior to the detergent lysis techniques. The samples were dialyzed against 0.1 M KCl, 0.01 M phosphate. The chromosomes were then labeled with the fluorescent or paramagnetic anti-dsDNA antibodies and sorted. The released chromosomes were labeled with the fluorescent scFv targeting dsDNA (f*scFv_{dsDNA}) and sorted one chromosome per well.

The locus for the epidermal growth factor receptor coding sequence was labeled by the fluorescent in situ hybridization (FISH) with the probe designed based upon the sequence from the GenBank (NCBI, NIH, Bethesda, Md.) with Lasergene (DNA Star, Madison, Wis.). It was synthesized on 380 A DNA Synthesizer (ABI, Foster City, Calif.). The probe was modified by terminal transferase with digoxigenin dNTP (Hoffmann-La Roche, Basel, Switzerland). After hybridization the chromosomes were labeled and amplified with the scFv targeting digoxigenin and chelating Eu (radiating red fluorescence upon multi-photon excitation). The centromeres were labeled by FISH with the probe modified with biotin, which was designed and synthesized as the one described above. After completion of hybridization, the chromosomes were labeled and amplified with the scFv chelating Tb (radiating green fluorescence). The chromosomes were anchored to the glass slide after they were first incubated with 1% amino-propyl-3ethoxy-silane at 60 deg C. and thereafter treated with 1% glutaraldehyde. Finally, the chromosomes mounted in PVP were imaged upon excitation with the multi-photon, tunable Ti:Al₂O₃ pulsed-laser or xenon source mounted on the inverted microscope (Zeiss, Oberkochen, Germany or Nikon. Tokyo, Japan).

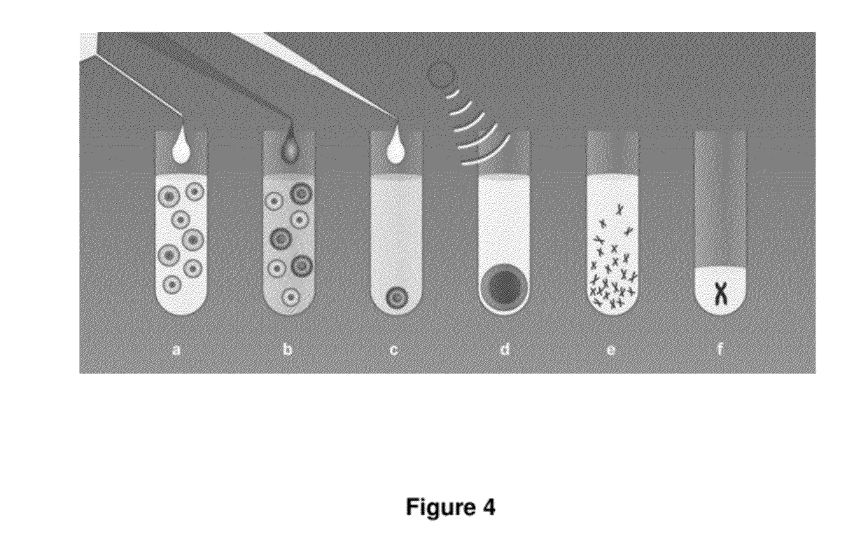

The steps of the procedure are illustrated in FIG. 4. Peritoneal washings, fine needle biopsies of the pelvic mass, cell cultures, or tissues from laparoscopies of the patients suspected of developing ovarian cancers were the sources of the cells (FIG. 4a). The cells were labeled with the genetically engineered, single chain variable fragment antibodies (scFv) targeting variant III of the epidermal growth factor receptor (EGFRvIII). These variants were present on cancer cells only, but entirely absent on healthy cells. The scFvs were rendered fluorescent (f*) or superparmagnetic (s*) by incorporating metal ions into the metal binding domains of the scFvs (FIG. 4b). That was followed by separation of the cells, rendered fluorescent or magnetic, by fluorescent activated cell sorting (FACS) or magnetically activated cell sorting (MACS). The sorting outcome was one cell with the defined molecular profile per well (FIG. 4c). Further steps involved their clonogenic propagation in vitro, synchronizing the cycles, or immediate tests. The tests involved immune-blotting after cell lysis and native electrophoresis or targeted polymerase chain reaction after total RNA isolation, reverse transcription, amplification, and sequencing. This step facilitated correlations between the molecular profiles of the selected cells with these specific cells' transcriptomes. In preparation for chromosome isolation, the cells were swollen in hypotonic solutions (FIG. 4d). The burst of low energy sonic radiation broke the cell membrane and skeletons releasing chromosomes (FIG. 4e). Through dialysis, the free chromosomes were purified from the cellular debris and immersed in the buffer solutions suitable for labeling with chromosome specific, fluorescent or superparamagnetic scFv targeting dsDNA. Whole, single, intact chromosomes were isolated one chromosome per well by sorting with the FACS or MACS instruments (FIG. 4e). Their integrities were inspected with molecular imaging and for sequencing.

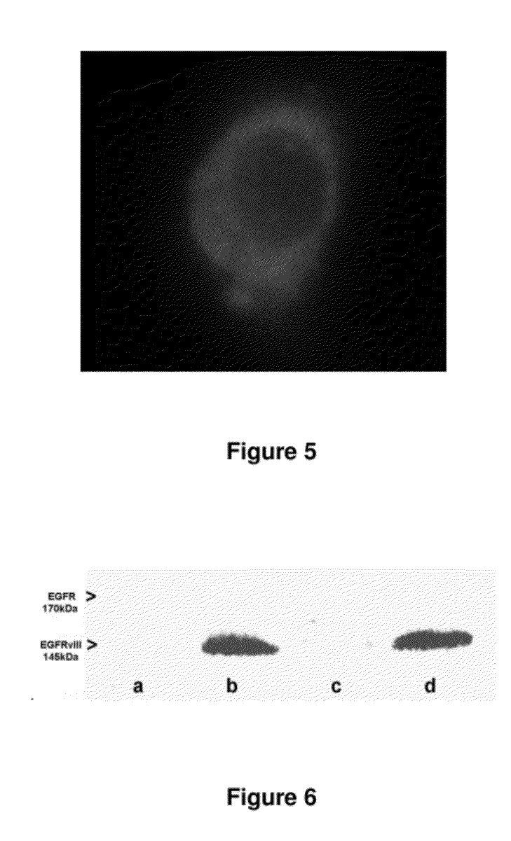

An example of an EGFRvIII positive cell from the peritoneal washing of the patient with the ovarian cancer, which was labeled with fluorescent scFv targeting EGFRvIII (scFv_{EGFRvIII}) and imaged with multi-photon excitation, pulsed laser fluorescence microscopy, is illustrated in FIG. 5. The cells obtained from the patients were labeled with scFv targeting EGFRvIII (scFv_{EGFRvIII}) and scFv_{EGFRwt}. The labeled cells were separated from all the other cells and sorted individually into wells by FACS or MACS. After labeling and sorting, this selected cell was transferred from the collector well into the environmental chamber of the multi-photon station for imaging in living state. The labeling was very specific, so no healthy cells were labeled, thus no healthy cells were sorted into the population of cancer cells. The metal binding domains of the scFv chelated Eu. This provided a very stable and strong fluorescence at the 619 nm upon three-photon or epifluorescence excitation. As shown, the scFv was effectively entering the cell, thus becoming a permanent marker of the cancer cell. Labeling with the stable biotags and the biotags' internalization assured the permanently present, strong, and stable fluorescence with the strengthened signal to noise ratio. The pulsed laser excitation reduced photo-toxicity and fading, thus enhanced quality of the subcellular imaging.

The specificity of the scFv targeting EGFRvIII on the ovarian cancer cells was also confirmed with immuno-blotting as illustrated in the FIG. 6. The selected cells were lysed and sonicated. Thereafter, the cells were either electrophoresed or the native receptors subjected to immuno-precipitation and native electrophoresis. Either way, the samples were labeled with scFv_{EGFRvIII}. The gene expression products from the DNA plasmids constructs, which were received from ATCC, coding wild type and truncated gene expression products served as the type III mutant negative (FIG. 6a) and positive controls (FIG. 6b). In the ovarian cancer cells, the expression of the wild type EGFR resulted in the complete absence of labeling with the scFv_{EGFRvIII} (FIG. 6c) identical to the negative control. The expression of the EGFRvIII mutation was immediately identified with the f*scFv_{EGFRvIII} (FIG. 6d). The presence of these controls facilitated an easy identification of wild type or mutated gene expression products in the ovarian cancer cells.

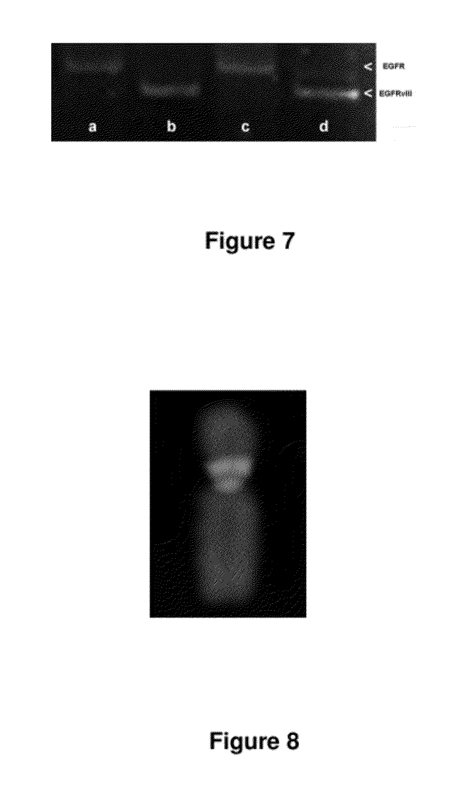

The profiles of the molecules displayed on the ovarian cancer cells were defined with the scFvs. Thereafter, the transcriptomes of these cells were probed by reverse transcription, amplification, and sequencing. The electrophoresed amplicons from the truncated transcripts are illustrated in the FIG. 7. Reverse transcription and polymerase chain reaction (RT PCR) driven amplification of the targeted sequences revealed differences in the sizes of amplicons. The amplicons from the DNA plasmids constructs, received from ATCC, coding wild type and truncated gene expression products, served as the type III mutant negative or wild type (FIG. 7a) and positive controls (FIG. 7b). In the transcripts from the isolated ovarian cancer cells, the receptors from the wild type transcripts resulted in the complete transcript sequence amplicons (FIG. 7c). However, the shorter amplicons resulted from the RT PCR of the truncated version of cDNA generated from the shorter, than the sequence in the wild type, transcript (FIG. 7d). The real lengths obviously were contingent upon the designed primers. Sequencing of these amplicons confirmed their homology with the sequences published in the public domain of the GenBank.

After these three steps of validation and selection of the ovarian cancer cells only, based upon the cancer specific biomarker, the sorted cancer cells were swollen and disrupted. These procedures released chromosomes, which were then dialyzed, labeled with the fluorescent scFv targeting dsDNA, and sorted one chromosome per well using FACS (Malecki 1996). Alternatively, they were labeled with the superparamagnetic, single chain variable fragment antibodies (s*scFv) and sorted with magnetically activated cell sorter (MACS). The individual chromosomes could then be selected one chromosome per well and hybridized with the sequence specific probes as illustrated in FIG. 8. This chromosome's centromere was subjected to fluorescent in situ hybridization (FISH). The chromosome 7 centromere specific probe was modified with the biotin. After completion of hybridization, the chromosome was labeled with the anti-biotin, genetically engineered, single chain variable fragment antibody (Malecki et al 2002). This scFv antibody contained Tb chelating domain. After its saturation with Tb, the scFv rendered the strong and stable green fluorescence. This was completed with the probe modified with the scFv targeting biotin and chelating Tb (green marker). If the probe was substituted with the dNTPs modified with digoxigenin, then the anti-dig scFvs were at work. The locus for the coding sequence of the epidermal growth factor receptor consensus coding sequence was labeled with the specific probe designed with the Primer 3 based upon the sequence from the GenBank. The probe was modified with modified with the digoxigenin. After completion of hybridization, the chromosome was labeled with the anti-digoxigenin, genetically engineered, single chain variable fragment antibody (Malecki et al. 2002). The metal binding domain of these scFvs chelated Eu. This was generating the strong and stable red fluorescence upon multi-photon excitation with the tunable laser. Upon completion of hybridization, the chromosome was anchored to the glass slide through amino-propyl-3ethoxy-silane binding resulting in exceptionally clean background. The chromosome was imaged with the epifluorescence and/or multi-photon excitation by the tunable, pulsed laser excitation. The chromosome preserved its integrity very well. Access of the probes to the sequences assured the strong and uniform hybridization. Organometallic clusters of the modified scFvs assured stable and efficient fluorescence.

Development, invasion, and metastasis of cancer are extremely complex processes. They involve multiple mutations in many genes and alteration within gene expression products. Although resulting in similar outcomes, being uncontrolled proliferations and spreading, these mutations may occur in multiple combinations. As only 1.5% of the genome is coding the proteins, thus is available for analysis as transcriptomes and proteomes. However, the remaining non-coding or junk DNA sequences play significant roles in carcinogenesis. Therefore, for a complete analysis of the processes involved in carcinogenesis in the clones of the cancer cells present in the tumors of all the patients, sequencing of the whole, complete genomes is really necessary. Thus, total RNA was isolated, reverse transcribed, and sequenced cDNA. Further, from these defined and isolated cells, whole intact chromosomes were isolated using a superparamagnetic or fluorescent scFv. The advantage of sorting the chromosomes with the aid of the fluorescently or magnetically modified scFv relies upon the fact that the scFv are easily separated from the genomic DNA prior to sequencing, while leaving no traces of fluorochromes. Moreover, in situ hybridization in liquid phase, as pursued here, provides a uniform access to the genes, which is often compromised in conventional FISH techniques due to firm adhesion to the slides after drying. Anchoring of the hybridized chromosomes after completion of FISH to the clean glass slides resulted in the exquisitely clean background, thus improved signal to noise ratio.

The chromosomes isolated from the cells of the defined molecular biomarker profile can be sequenced either with the traditional Sanger method or with the next generation massive parallel sequencers capable for the long reads, while accelerating the read-out times, improving their specificity, and validating annotations based sequence assignments.

### Example 6

### Molecular Imaging in Mice and Rats: Au*Death Tags Highlight Dead Cells in Tumors In Vivo

Nude mice carrying spontaneously growing tumors or injected on the shoulder with cancer cells (xenografts), were studied. The cancer was treated with a antioxidant enzyme blocker that induces cancer cell suicide (e.g., antiSOD, anti-Gpx, anti-caspase or a combination thereof). Effectiveness of the therapy was evaluated by intra-tail-vein injection of death tags and imaged with fluorescence. FIG. 3 shows a nude mouse suffering from cancer and treated with cancer suicide therapy as described. The body heat was detected and reveals the body anatomy and the death tags that label dead cells are shown with the green fluorescence channel (indicated by arrow), highlighting the cancer cells that are dead due to the gene therapy. In this case, the cancer therapy is effective because the induction of apoptosis by the therapy resulted in death of the cancer cells. Similar results may be obtained using a chemotherapeutic or other systemic therapy that induces apoptosis.

Effective and lethal dose determinations. Having approved IACUC protocols, the mice and rats were injected via tail veins with increasing concentrations of death tags tagged with Au nanoparticles in single or multiple bolus of up to 3M molarity. There were no effects on their behavior or life span.

### REFERENCES

Bauer R D, Johanson R. A gentle method for isolation of DNA and histones from the same small tissue sample. Biochim Biophys Acta. 1966 May 19; 119(2):418-20.
Chomczynski P, Sacchi N. Single-step method of RNA isolation by acid guanidinium thiocyanate-phenol-chloroform extraction. Anal Biochem. 1987 April; 162(1):156-9.
Curtet C, Tellier C, Bohy J, Conti M L, Saccavini J C, Thedrez P, Douillard J Y, Chatal J F, Koprowski H. Selective modification of NMR relaxation time in human colorectal carcinoma by using gadolinium-diethylenetriaminepentaacetic acid conjugated with monoclonal antibody 19-9. Proc Natl Acad Sci USA 1986; 83(12):4277-4281.
Deo A, Fogel M, Cowper S E. Nephrogenic systemic fibrosis: a population study examining the relationship of disease development to gadolinium exposure. Clin J Am Soc Nephrol. 2007 March; 2(2):264-7. Epub 2007 Feb. 7.
Déret S, Maissiat C, Aucouturier P, Chomilier J. SUBIM: a program for analysing the Kabat database and determining the variability subgroup of a new immunoglobulin sequence. Comput Appl Biosci. 1995 August; 11(4):435-9.
Gatewood J M, Cook G R, Balhorn R, Schmid C W, Bradbury E M. Isolation of four core histones from human sperm chromatin representing a minor subset of somatic histones. The Journal of Biological Chemistry. 1990, 265, 20662-20666.
Jemal A, Siegel R, Xu J, Ward E. Cancer Statistics 2010. CA Cancer J Clin 2010; 60; 277-300.
Johnson G, Wu T T. The Kabat database and a bioinformatics example. Methods Mol. Biol. 2004; 248:11-25.
Kobayashi H, Kawamoto S, Jo S K, Bryant H L Jr, Brechbiel M W, Star R A. Macromolecular MRI contrast agents with small dendrimers: pharmacokinetic differences between sizes and cores. Bioconjug Chem 2003; 14(2):388-394.
Linger E C, Totty W G, Neufeld D M, Otsuka F L, Murphy W A, Welch M S, Connett J M, Philpott G W. Magnetic resonance imaging using gadolinium labeled monoclonal antibody. Invest Radiol. 1985; 20(7):693-700.
Malecki M, Hsu A, Truong L, Sanchez S. Molecular immunolabeling with recombinant single-chain variable fragment (scFv) antibodies designed with metal-binding domains; Proc. Natl. Acad. Sci. USA, 2002, 99, 213-218.
Malecki M. 1996. Preparation of plasmid DNA in transfection complexes for spectroscopic imaging. In: Marek Malecki and Godfried Roomans (eds.): Specimen Preparation for Microscopy and Microanalysis. SM International Press, Chicago, Ill.: 1-16.
Mendonca Dias M H, Lauterbur P C. Ferromagnetic particles as contrast agents for magnetic resonance imaging of liver and spleen. Magn Reson Med 1986; 3(2):328-330.
McKoy J M, Laumann A, Samaras A, Lacouture M, West D, Raisch D, Obadina E, Fajolu O, Reilly L, Bennett C L. Gadolinium-associated nephrogenic systemic fibrosis. Community Oncol. 2008; 5(6):325-326.
National Highway Traffic Safety Administration Data Resource Website (http://www-fars.nhtsa.dot.gov/Main/index.aspx)
Roberts R W, Szostak J W. RNA-peptide fusions for the in Vitro selection of peptides and proteins. Proc. Natl. Acad. Sci. USA 1997; 94: 12297-12302.
Shoemann R L, Traub P. The in vitro DNA-binding properties of purified nuclear lamin proteins and vimentin. J. Biol. Chem. 1990, 265, 9055-9061.
Unger E C, Shen D, Wu G, Stewart L, Matsunaga T O, Trouard T P. Gadolinium-containing copolymeric chelates—a new potential MR contrast agent. MAGMA 1999; 8(3):154-162.
Weissleder R. Target-specific superparamagnetic MR contrast agents. Magn Reson Med 1991; 22(2):209-212; discussion 213-215.Wilson D S, Keefe A D, Szostak J W. The use of mRNA display to select high-affinity protein-binding peptides. Proc Natl Acad Sci USA. 2001 Mar. 27; 98(7):3750-5.
Vanden, T. “Afghan insurgents match surge with more IEDs”. USA Today, Jan. 10, 2011. http://www.usatoday.com/news/military/2011-01-10-ieds10_ST_N.htm.; retrieved 2011 Jun. 13.

What is claimed is:

1. A death tag for targeting a cell death marker comprising:
a death marker binding domain;
a reporter binding domain (RBD); and
a reporter component that is associated with the reporter binding domain.

2. The death tag of claim 1, wherein the death marker is indicative of terminal cell death.

3. The death tag of claim 2, wherein the death marker is genomic DNA, single-stranded DNA, double stranded DNA, histones, lamins, IF, cell cytoskeletal molecules, nuclear matrix molecules, contractile molecules or fragments thereof.

4. The death tag of claim 1, wherein the death marker is indicative of early apoptosis.

5. The death tag of claim 2, wherein the death marker is phosphatidyl serine.

6. The death tag of claim 1, wherein the death marker binding domain is an scFv, sdFv, SDR, CDR, CD, IgG, IgM or functional fragments thereof.

7. The death tag of claim 1, wherein the reporter binding domain is a metal binding domain.

8. The death tag of claim 1, wherein the reporter component is a metal ion or nanoparticle.

9. The death tag of claim 7, wherein the metal nanoparticle or atoms are selected from Au, Pt, Pd and Ag.

10. The death tag of claim 8, wherein the metal ion or nanoparticle is a superparamagnetic metal.

11. The death tag of claim 10, wherein superparamagnetic metal is Gd, Eu, Fe, Ni or Co.

12. The death tag of claim 9, wherein the metal nanoparticle tag is a core-shell nanoparticle.

13. The death tag of claim 1, wherein the reporter binding domain is a fluorochrome binding domain or functional group.

14. The death tag of claim 1, wherein the reporter component is a fluorochrome.

15. The death tag of claim 14, wherein the fluorochrome having a visible, UV or infrared wavelength, and is detected through the use of Stoke's, Raman, or fluorescence.

16. The death tag of claim 1, wherein the reporter binding domain is a radionuclide binding domain or functional group.

17. The death tag of claim 1, wherein the reporter component is a radionuclide.

18. The death tag of claim 17, wherein the radionuclide is ^{99m}Tc, ¹²⁵I, ¹¹¹In, ¹²³I, ¹³¹I, ¹⁸F or ⁶⁴Cu.

19. The death tag of claim 1, wherein the reporter binding domain is a microbubble binding domain or functional group.

20. The death tag of claim 1, wherein the reporter component is a microbubble.

21. The death tag of claim 19, wherein the microbubble is characterized by its response to ultrasound.

22. The death tag of claim 1, wherein the death tag can be used to detect the extent of cell death resulting from cancer or toxic cancer treatment, myocardial infarction, stroke, frost, heat, or ischemia, traffic accidents, blunt force trauma, accident crashes, sporting accidents, or improvised explosive devices.

23. The death tag of claim 1, wherein the death tag can be used to target, detect, and remove circulating free DNA, histones, lamins or a combination thereof, from a physiological fluid in the subject.

24. A method of determining the need for or the efficacy of a treatment comprising:
administering to a subject an effective dose of a death tag that targets apoptotic, necrotic or dead cells;
exposing the subject to an imaging technique;
determining that the treatment is effective when the imaging technique detects the presence or change in the amount of the death tag.

25. The method of claim 24, wherein the death tag comprises a death marker binding domain, a reporter binding domain (RBD), and a reporter component that is associated with the reporter binding domain.

26. The method of claim 24, wherein the death marker is phosphatidyl serine, genomic DNA, single-stranded DNA, double stranded DNA, histones, lamins, cell cytoskeleton molecules, contractile molecules or fragments thereof.

27. The method of claim 24, wherein the death marker binding domain is an scFv, sdFv, CDR, SDR, CD, Fab or a functional fragment thereof.

28. The method of claim 24, wherein the reporter component is a noble metal nanoparticle selected from Au, Pt, Pd and Ag.

29. The method of claim 24, wherein the reporter component is a superparamagnetic metal nanoparticle selected from Gd, Eu, Fe, Ni, and Co.

30. The method of claim 24, wherein the reporter component is a core-shell nanoparticle, the core shell nanoparticle comprising an inner superparamagnetic metal core and an outer noble metal shell.

31. The method of claim 24, wherein the imaging technique is radiography, CT, MRI, NMR, USG, Fluorescence, IR, Raman, gammascintigraphy, SPECT or PET.
